(19) United States
(12) Patent Application Publication
(10) Pub. No.: US 2025/0186675 A1
(43) Pub. Date: Jun. 12, 2025

(54) IMPROVED DRESSINGS AND SYSTEMS FOR NEGATIVE-PRESSURE WOUND THERAPY AND INSTILLATION THERAPY HAVING LOW-VOLUME MANIFOLDS AND BYPASS INSTILLATION PASSAGES

(71) Applicant: Solventum Intellectual Properties Company, Maplewood, MN (US)

(72) Inventors: Benjamin A. PRATT, Bracknell (GB); Timothy M. ROBINSON, Bracknell (GB); Christopher BREACH, Saint Paul, MN (US)

(21) Appl. No.: 18/845,191

(22) PCT Filed: Feb. 24, 2023
(86) PCT No.: PCT/IB2023/051739

§ 371 Date: Sep. 9, 2024

Related U.S. Application Data
(60) Provisional application No. 63/318,150, filed on Mar. 9, 2022.

(51) Int. Cl. A61M 1/00; A61M 35/00

(52) U.S. Cl. CPC A61M 1/915; A61M 1/912; A61M 35/00; A61M 35/30; A61M 2202/0208

(57) ABSTRACT

A system for treating a tissue site with negative-pressure therapy includes a dressing configured to be placed adjacent to the tissue site. The dressing includes a first film layer, a manifold, and a second film layer. A plurality of fenestrations is disposed through a first side and a second side of the first film layer and is configured to be exposed to the tissue site. The first side of the manifold is disposed adjacent to the second side of the first film layer, and a first side of the second film layer is disposed adjacent to the second side of the manifold. A negative-pressure passage is disposed through the second film layer, and a fluid-ingress passage is disposed through the second film layer, the manifold, and the first film layer. A negative-pressure source is fluidly coupled to the negative-pressure passage, and a fluid source coupled to the fluid-ingress passage.

### CROSS-REFERENCE TO RELATED APPLICATIONS

This application is a U.S. National Stage Entry of International Application No. PCT/IB2023/051739, filed on Feb. 24, 2023, which claims the benefit of priority to U.S. Provisional Application No. 63/318,150, filed on Mar. 9, 2022, each of which are incorporated herein by reference in their entirety.

### TECHNICAL FIELD

The invention set forth in the appended claims relates generally to tissue treatment systems and more particularly, but without limitation, dressings and systems for treating wounds with negative-pressure wound therapy and instillation therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for detecting the presence of liquid in the fluid pathways of a negative-pressure therapy system are set forth in the appended claims. Illustrative embodiments are also provided to enable a person skilled in the art to make and use the claimed subject matter.

A system for treating a tissue site with negative-pressure therapy is presented. The system may include a dressing configured to be placed adjacent to the tissue site. The dressing may include a first film layer, a manifold, and a second film layer. The first film layer may have a first side opposite a second side, and a plurality of fenestrations may be disposed through the first side and the second side and configured to be exposed to the tissue site. The manifold may have a first side opposite a second side, and the first side of the manifold may be disposed adjacent to the second side of the first film layer. The second film layer may have a first side opposite a second side, and the first side of the second film layer may be disposed adjacent to the second side of the manifold. A negative-pressure passage may be disposed through the second film layer and in direct fluid communication between the second film layer and the manifold. A fluid-ingress passage may be disposed through the second film layer, the manifold, and the first film layer. The fluid-ingress manifold may be in direct communication with the first side of the first film layer. A negative-pressure source may be configured to be fluidly coupled to the negative-pressure passage, and a fluid source may be configured to be fluidly coupled to the fluid-ingress passage.

In other features, the first film layer may include a fluid-ingress aperture, the manifold may include a fluid-ingress aperture, and the second film layer may include a fluid-ingress aperture. The fluid-ingress aperture of the first film layer, the fluid-ingress aperture of the manifold, and the fluid-ingress aperture of the second film layer may be coaxially aligned to define the fluid-ingress passage through the fluid-ingress aperture of the first film layer, the fluid-ingress aperture of the manifold, and the fluid-ingress aperture of the second film layer. In other features, a portion of the second side of the first film layer around the fluid-ingress aperture of the first film layer may be coupled to a portion of the first side of the manifold around the fluid-ingress aperture of the manifold, and a portion of the first side of the second film layer around the fluid-ingress aperture of the second film layer may be coupled to a portion of the second side of the manifold around the fluid-ingress aperture of the manifold. In other features, a periphery of the second side of the first film layer is coupled to a periphery of the first side of the manifold, and a periphery of the first side of the second film layer is coupled to a periphery of the second side of the manifold.

In other features, a first chamber may be defined between the second side of the first film layer and the first side of the manifold, the portion of the second side of the first film layer coupled to the portion of the first side of the manifold, and the periphery of the second side of the first film layer coupled to the periphery of the first side of the manifold. A second chamber may be defined between the first side of the second film layer and the second side of the manifold, the portion of the first side of the second film layer coupled to the portion of the second side of the manifold, and the periphery of the first side of the second film layer coupled to the periphery of the first side of the manifold. In other features, the first chamber and the second chamber may be fluidly isolated from the fluid-ingress passage. In other features, the manifold may further include a plurality of windows formed through the manifold. In other features, the first chamber may be in fluid communication with the second chamber through the plurality of windows. In other features, the first side of the first film layer may be configured to be in direct contact with the tissue site. In other features, the fluid-ingress passage may be configured to be in direct fluid communication with the tissue site.

In other features, the second film layer may include a negative-pressure aperture. The negative-pressure passage may be defined by a fluid pathway from the negative-pressure aperture to the second chamber, and the negative-pressure passage may be configured to be positioned in fluid communication with the tissue site through the plurality of windows, the first chamber, and the plurality of fenestrations. In other features, the dressing may further include a sealing layer having a treatment aperture and a plurality of bonding apertures around the treatment aperture. The first side of the film layer and the plurality of fenestrations may be configured to be exposed to the tissue site through the treatment aperture. In other features, the dressing may further include a cover layer having a first side opposite a second side, and a pressure-sensitive adhesive disposed on the first side of the cover layer. The pressure-sensitive adhesive may couple the cover layer to the second film layer and the sealing layer. A portion of the pressure-sensitive adhesive may be disposed adjacent to the plurality of bonding apertures.

In other features, the first film layer may include thicker portions between each fenestration of the plurality of fenestrations and thinner portions proximate each fenestration of the plurality of fenestrations. In other features, the second layer may be substantially coextensive with the manifold and the first film layer. In other features, the second film layer may be substantially coextensive with the sealing layer. In other features, a plurality of standoffs may be formed on one or both of the first side and the second side of the manifold. In other features, the manyfold may include an open-cell foam. In other features, the dressing may further include a fluid-ingress cover configured to be removably coupled to the second side of the second film layer. In other features, the fluid source may be configured to provide instillation solution to the dressing. In other features, the fluid source may be an oxygen source configured to provide oxygen to the dressing. In other features, the manifold may be fluidly isolated from the fluid-ingress passage.

A system for treating a tissue site with negative-pressure therapy is also presented. The system may include a dressing configured to be positioned adjacent to the tissue site. The dressing may include a first film layer, a manifold, and a second film layer. The first film layer may include a plurality of fenestrations and a fluid-ingress aperture. The manifold may include a plurality of windows and a fluid-ingress aperture. The second film layer may include a negative-pressure aperture and a fluid-ingress aperture. The first film layer, the manifold, and the second film layer may be assembled in a stacked relationship to define a first chamber between the first film layer and the manifold and a second chamber between the second film layer and the manifold. The fluid-ingress aperture of the first film layer, the fluid-ingress aperture of the manifold, and the fluid-ingress aperture of the second film layer may define a fluid-ingress passage through the dressing. The first chamber and the second chamber may be fluidly isolated from the fluid-ingress passage.

In various implementations, the system may include a fluid source coupled to the fluid-ingress passage through the fluid-ingress aperture of the second film layer. The fluid source may be configured to be in direct fluid communication with the tissue site through the fluid-ingress passage. In various implementations, the system includes a negative-pressure source coupled to the negative-pressure aperture of the second film layer. The negative-pressure source may be configured to provide negative-pressure to the tissue site through the plurality of fenestrations of the first film layer, the first chamber, the plurality of windows of the manifold, the second chamber, and the negative-pressure aperture of the second film layer.

In other features, the system may include a sealing layer, which includes a treatment aperture and a plurality of bonding apertures around the treatment aperture. The first film layer may be configured to be coupled to the sealing layer such that the plurality of fenestrations of the first film layer are exposed through the treatment aperture. In other features, the system may include a cover that includes a pressure-sensitive adhesive. The cover may be coupled to the sealing layer around the manifold. At least a portion of the pressure-sensitive adhesive may be exposed through the bonding aperture. In other features, the second film layer may be substantially coextensive with the sealing layer. In other features, the first film layer, the manifold, and the second film layer may be substantially coextensive with each other.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment and instillation treatment in accordance with this specification;

FIG. 2 is an exploded view of an example of the dressing of FIG. 1, illustrating additional details that may be associated with some embodiments suitable for use with both negative-pressure wound therapy and instillation therapy and/or oxygen therapy;

FIG. 3 is an isometric view of the dressing of FIG. 2, as assembled;

FIG. 4 is an isometric view of the dressing of FIG. 2, as assembled, and with dressing interfaces attached;

FIG. 5 is a top view of an assembled dressing according to this specification;

FIG. 6 is a bottom view of an assembled dressing according to this specification;

FIG. 7 is a top view illustrating additional details, which may be associated with some examples of a dressing according to this specification;

FIG. 8 is a top view illustrating additional details, which may be associated with some examples of a dressing according to this specification;

FIG. 9 is a top view illustrating additional details, which may be associated with some examples of a dressing according to this specification;

FIG. 10 is a top view illustrating additional details, which may be associated with some examples of a dressing according to this specification;

FIG. 11 is a top view illustrating additional details, which may be associated with some examples of a dressing according to this specification;

FIG. 12 is a top view illustrating additional details, which may be associated with some examples of a dressing according to this specification;

FIG. 13 is a top view illustrating additional details, which may be associated with some examples of a dressing according to this specification;

FIG. 14 is a top view illustrating additional details, which may be associated with some examples of a dressing according to this specification;

FIG. 15 is a top view illustrating additional details, which may be associated with some examples of a dressing according to this specification;

FIG. 16 is a top view illustrating additional details, which may be associated with some examples of a dressing according to this specification;

FIG. 17 is a top view illustrating additional details, which may be associated with some examples of a dressing according to this specification;

FIG. 18 is a top view illustrating additional details, which may be associated with some examples of a dressing according to this specification;

FIG. 19 is a cross-sectional view of the example dressing of FIG. 4, taken at line 19-19, applied to an example tissue site, and illustrating additional details associated with some examples of the therapy system of FIG. 1;

FIG. 19A is a detail view, taken at reference 19A in FIG. 19, illustrating additional features, which may be associated with some examples of the dressing of FIG. 19;

FIG. 19B is a detail view illustrating additional features, which may be associated with the detail view of FIG. 19A in some implementations of the dressing of FIG. 19;

FIG. 19C is a detail view, taken at reference 19C in FIG. 19, illustrating additional features, which may be associated with some example embodiments of the dressing of FIG. 19;

FIG. 19D is a detail view illustrating additional features, which may be associated with the detail view of FIG. 19C in some implementations of the dressing of FIG. 19;

FIG. 19E is a cross-sectional view illustrating additional details, which may be associated with some examples of the dressing of FIG. 19 applied to a tissue site having a contour;

FIG. 19F is a cross-sectional view illustrating additional details, which may be associated with some examples of the dressing of FIG. 19 applied to a tissue site having a contour;

FIG. 20 is an exploded view of an example of the dressing of FIG. 1, illustrating additional details, which may be associated with some embodiments;

FIG. 21 is an isometric view of an assembled example of the dressing of FIG. 20 with dressing interfaces for negative-pressure wound therapy and instillation and/or oxygen therapy attached;

FIG. 22 is a cross-sectional view of the example dressing of FIG. 21, taken at line 22-22, applied to an example tissue site, and illustrating additional details associated with the therapy system of FIG. 1;

FIG. 23 is an exploded view of an example of the dressing of FIG. 1, illustrating additional details that may be associated with some embodiments;

FIG. 24 is an isometric view of an assembled example of the dressing of FIG. 23; and

FIG. 25 is a cross-sectional view of the example dressing of FIG. 24, taken at line 25-25, and applied to an example tissue site.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

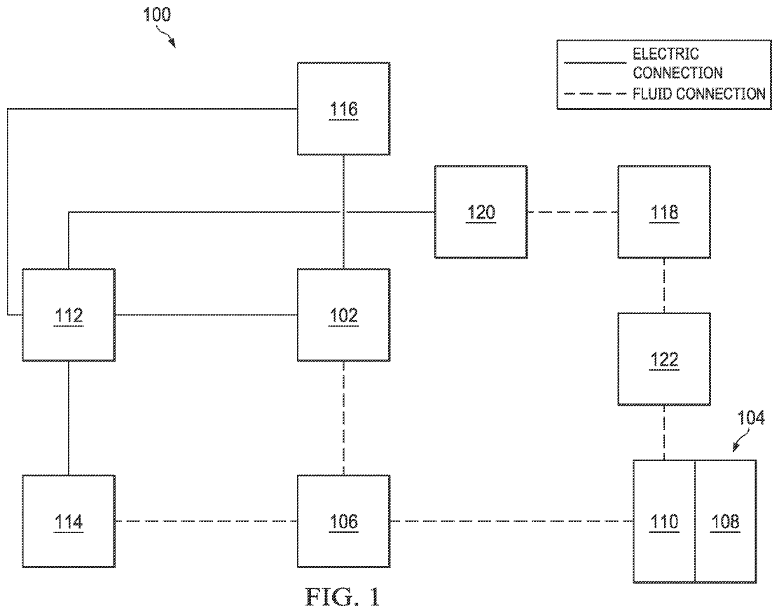

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Texas.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source such as a positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 118, and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, may be any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, for example, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid through the tissue interface 108 under pressure. For example, a manifold may be adapted to receive negative pressure from a source and distribute negative pressure through multiple apertures through the tissue interface 108, which may have the effect of collecting fluid from a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S. A., Colombes, France; and Inspire 2301 and Inspire 2327 polyurethane films, commercially available from Transcontinental Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

In some embodiments, the controller 112 may have a continuous pressure mode, in which the negative-pressure source 102 is operated to provide a constant target negative pressure for the duration of treatment or until manually deactivated. Additionally or alternatively, the controller may have an intermittent pressure mode. In example embodiments, the controller 112 can operate the negative-pressure source 102 to cycle between a target pressure and atmospheric pressure. For example, the target pressure may be set at a value of 135 mmHg for a specified period of time (e.g., five minutes), followed by a specified period of time (e.g., two minutes) of deactivation. The cycle can be repeated by activating the negative-pressure source 102, which can form a square wave pattern between the target pressure and atmospheric pressure.

In some example embodiments, the increase in negative-pressure from ambient pressure to the target pressure may not be instantaneous. For example, the negative-pressure source 102 and the dressing 104 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise rate of negative pressure set at a rate of 25 mmHg/min. and a descent rate set at 25 mmHg/min. In other embodiments of the therapy system 100, the triangular waveform may vary between negative pressure of 25 and 135 mmHg with a rise rate of about 30 mmHg/min. and a descent rate set at about 30 mmHg/min.

In some embodiments, the controller 112 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 112, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 112 may receive and process data, such as data related to instillation solution provided to the tissue interface 108. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 112 may also control the operation of one or more components of the therapy system 100 to instill solution. For example, the controller 112 may manage fluid distributed from the solution source 118 to the tissue interface 108. In some embodiments, fluid may be instilled to a tissue site by applying a negative pressure from the negative-pressure source 102 to reduce the pressure at the tissue site, drawing solution into the tissue interface 108. In some embodiments, solution may be instilled to a tissue site by applying a positive pressure from the positive-pressure source 160 to move solution from the solution source 118 to the tissue interface 108. Additionally or alternatively, the solution source 118 may be elevated to a height sufficient to allow gravity to move solution into the tissue interface 108.

The controller 112 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 108, or it may be implemented to provide a dynamic pressure mode of operation to vary the flow rate of instillation solution through the tissue interface 108. Alternatively, the application of negative pressure may be implemented to provide an intermittent mode of operation to allow instillation solution to dwell at the tissue interface 108. In an intermittent mode, a specific fill volume and dwell time may be provided depending, for example, on the type of tissue site being treated and the type of dressing being utilized. After or during instillation of solution, negative-pressure treatment may be applied. The controller 112 may be utilized to select a mode of operation and the duration of the negative pressure treatment before commencing another instillation cycle by instilling more solution.

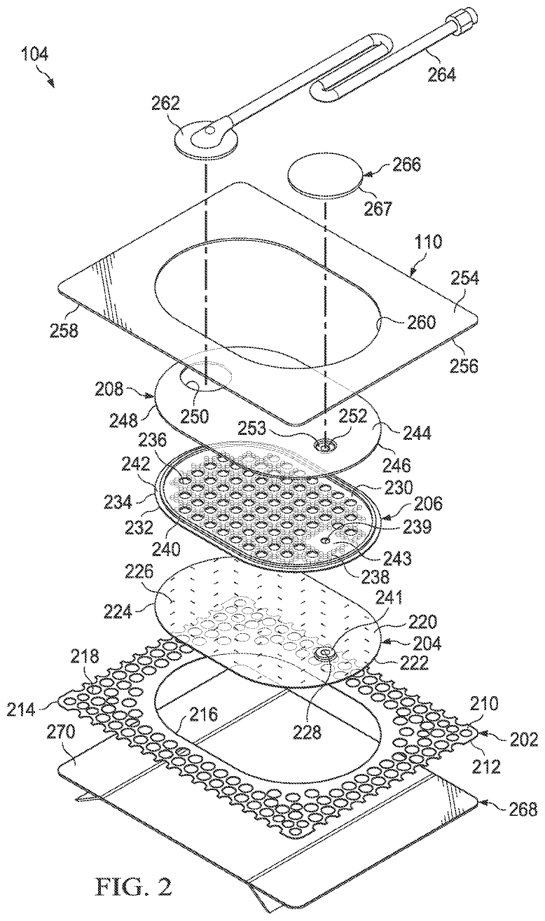

FIG. 2 is an exploded view of an example of the dressing 104 of FIG. 1, illustrating additional details that may be associated with some embodiments for use with both negative-pressure wound therapy and instillation therapy and/or oxygen therapy. In the example of FIG. 2, the dressing 104 may include a sealing layer 202, a first film layer 204, a manifold layer 206, a second film layer 208, and the cover 110. The sealing layer 202 may be formed from a soft, pliable material suitable for providing a fluid seal with a tissue site, such as a suitable gel material, and may have a substantially flat surface. In various implementations, the sealing layer 202 may include, without limitation, a silicone gel, a soft silicone, hydrocolloid, hydrogel, polyurethane gel, polyolefin gel, hydrogenated styrenic copolymer gel, a foamed gel, soft closed-cell foams such as polyurethanes and polyolefins coated with adhesives, polyurethane, polyolefin, or hydrogenated styrenic copolymers. In various implementations, the sealing layer 202 may have a thickness in a range of about 200 micrometers to about 1,000 micrometers. In various implementations, the sealing layer 202 may be formed from hydrophobic or hydrophilic materials.

In various implementations, the sealing layer 202 may be include or be formed from a hydrophobic or hydrophobic-coated material. For example, the sealing layer 202 may be formed by coating a spaced material, such as woven, nonwoven, molded, or extruded mesh, with a hydrophobic material such as a soft silicone.

The sealing layer 202 may have a top surface 210 opposite a bottom surface 212, a periphery 214 defined by an outer perimeter of the sealing layer 202, and a treatment aperture 216 formed through the sealing layer 202. In various implementations, the treatment aperture 216 may have an outline complementary to or corresponding to an outer perimeter of the manifold 206. The sealing layer 202 may also include a plurality of apertures 218 formed through the sealing layer 202. In various implementations, the plurality of apertures 218 may be formed through a region of the sealing layer 202 between the treatment aperture 216 and the periphery 214.

In various implementations, the apertures 218 may be formed by cutting, perforating, or applying local radio-frequency or ultrasonic energy through the sealing layer 202. In various implementations, the apertures 218 may be formed by other suitable techniques for forming an opening or perforation in the sealing layer 202. In various implementations, the apertures 218 may have a uniform distribution pattern, or may be randomly distributed. In various implementations, the apertures 218 may have many any combination of shapes, including circles, squares, stars, ovals, polygons, slits, complex curves, rectilinear shapes, or triangles.

In various implementations, each of the apertures 218 may have uniform or similar geometric properties. For example, each of the apertures 218 may be a circular aperture, and have substantially the same diameter. In various implementations, each of the apertures 218 may have a diameter in a range of between about 1 millimeter and about 20 millimeters.

In various implementations, the geometric properties of the apertures 218 may vary. For example, the diameters of the apertures 218 may vary depending on the positioning of the respective apertures 218 in the sealing layer 202. In various implementations, at least some of the apertures 218 may have a diameter in a range of between about 5 millimeters to about 10 millimeters. In various implementations, at least some of the apertures 218 may have a diameter in a range of between about 7 millimeters and about 9 millimeters. In various implementations, the sealing layer 202 may include corners, and the apertures 218 disposed at or near the corners may have diameters in a range of between about 7 millimeters and about 8 millimeters.

In various implementations, at least some of the apertures 218 positioned near the periphery 214 may have an interior that is cut open or exposed at the periphery 214 and is in lateral communication in a lateral direction (relative to the top surface 210 and/or bottom surface 212) with the periphery 214. In various implementations, the lateral direction may refer to a direction in a same plane as the top surface 210 and/or bottom surface 212 and extending towards the periphery 214. In various implementations, at least some of the apertures 218 positioned proximate to or at the periphery 214 may be spaced substantially equidistantly around the periphery 214. Alternatively, in various implementations, the spacing of the apertures 218 proximate to or at the periphery 214 may be spaced irregularly.

The first film layer 204 may include a suitable structure for controlling or managing fluid flow. In various implementations, the first film layer 204 may be a fluid-control layer that includes a liquid-impermeable, vapor-permeable elastomeric material. In various implementations, the first film layer 204 may be formed from or include a polymer film. For example, in various implementations, the first film layer 204 may be formed from or include a polyolefin film, such as a polyethylene film. In various implementations, the first film layer 204 may be substantially clear or optically transparent. In various implementations, the first film layer 204 may be formed from or include the same material as the cover 110. In various implementations, the first film layer 204 may be formed from or include a biocompatible polyurethane film tested and certified according to the USP Class VI Standard. In various implementations, the first film layer 204 may also have a smooth or matte surface texture. In various implementations, the first film layer 204 may have a glossy or shiny finish equal to or exceeding a grade B3 according to the Society of Plastics Industry (SPI) standards. In various implementations, the surface of the first film layer 204 may be a substantially flat surface, with height variations in a range of about 0.2 millimeters to about 1 centimeter.

In various implementations, the first film layer 204 may be hydrophobic. The hydrophobicity of the first film layer 204 may vary, but may have a contact angle with water of at least 90 degrees in some examples. In various implementations, the first film layer 204 may have a contact angle with water of no more than 150 degrees. In various implementations, the first film layer 204 may have a contact angle with water in a range of about 90 degrees to about 120 degrees, or in a range of about 120 degrees to about 150 degrees. Water contact angle may be measured using any standard apparatus. Although manual goniometers may be used to visually approximate contact angles, contact angle measuring instruments may often involve integrated systems that include a level stage, a liquid dropper (such as a syringe), a camera, and software designed to calculated contact angles more accurately and precisely. Non-limiting examples of such integrated systems include the FTÅ125, FTÅ200, FTÅ2000, and FTÅ4000 systems, all commercially available from First Ten Angstroms, Inc., of Portsmouth, Virginia, and the DTA25, DTA30, and DTA100 systems, all commercially available from Kruss GmbH of Hamburg, Germany. Unless otherwise specified, water contact angles herein are measured using deionized and/or distilled water on a level sample surface for a sessile drop added from a height of no more than five centimeters in air at 20-25° C. and 20-50% relative humidity. Contact angles herein represent averages of five to nine measured values, with the highest and lowest measure values discarded. In various implementations, the hydrophobicity of the first film layer 204 may be further enhanced with a hydrophobic coating of other materials, such as silicones and fluorocarbons.

The first film layer 204 may also be suitable for welding to other layers, including the manifold layer 206 and the second film layer 208. In various implementations, the first film layer 204 may be adapted for welding to polymers such as polyurethane, polyurethane films, and polyurethane foams using heat welding, radio-frequency (RF) welding, ultrasonic welding, or other methods. RF welding may be particularly suitable for more polar materials, such as polyurethane, polyamides, polyesters, and acrylates. Sacrificial polar interfaces may be used to facilitate RF welding of less polar film materials, such as polyethylene.

The area density of the first film layer 204 may vary according to a prescribed therapy or application. In various implementations, an area density of less than 40 grams per square meter may be suitable. In various implementations, the area density of the first film layer 204 may be in a range of about 20 grams per square meter to about 30 grams per square meter.

In various implementations, the first film layer 204 may be formed from or include a hydrophobic polymer, such as a polyethylene film. The simple and inert structure of polyethylene provides a surface that interacts little, if any, with biological tissues and fluids, and provides a surface that may encourage the free flow of liquids and exhibits a low adherence to tissues and fluids, properties that may be particularly advantageous for many applications. In various implementations, the first film layer 204 may be formed from other polymeric films such as polyurethanes, acrylics, polyolefins (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate styreneics, silicones, fluoropolymers, and acetates. In various implementations, the first film layer 204 may have a thickness in a range of about 20 micrometers to about 500 micrometers. In various implementations, the first film layer 204 may have a thickness of about 23 micrometers, about 25 micrometers, about 100 micrometers, about 250 micrometers, about 300 micrometers, and about 500 micrometers. In various implementations, the first film layer 204 may include a polar film suitable for lamination to the polyethylene film, such as polyamides, co-polyesters, ionomers, and acrylics. In various implementations, the first film layer 204 may include a tie layer to improve the bond between the polyethylene and polar film layers. In various implementations, the tie layer may include ethylene vinyl acetate or modified polyurethanes. In various implementations, the first film layer 204 may include an ethyl methyl acrylate (EMA) film.

As illustrated in FIG. 2, the first film layer 204 may have a top surface 220 opposite a bottom surface 222, and a periphery 224 defined by an outer perimeter of the first film layer 204. In various implementations, the periphery 224 may be a stadium, discorectangular, or obround shape. The first film layer 204 may also include one or more fluid passages 226 formed through the first film layer 204, and which may be distributed uniformly or randomly across the first film layer 204. In various implementations, the first film layer 204 may include a fluid-ingress or instillation aperture, such as aperture 228.

In various implementations, the fluid passages 226 may function as bi-directional and fluid-responsive valves. For example, each fluid passages 226 may be an elastic passage that is normally unstrained to prevent or substantially reduce fluid flow across the fluid passage 226, and can expand or open to allow fluid flow across the fluid passage 226 in response to a pressure gradient applied across the fluid passage 226. In various implementations, the fluid passages 226 may include perforations formed in the first film layer 204. Perforations may be formed by removing material from the first film layer 204, or cutting through the first film layer 204. In various implementations, cutting through the first film layer 204 may deform the edges of the perforations. In various implementations, the fluid passages 226 may be sufficiently narrow to form a seal or a fluid restriction to substantially reduce or prevent fluid flow across the fluid passage 226, particularly in the absence of a pressure differential. In various implementations, one or more of the fluid passages 226 may be an elastomeric valve that is normally closed when unstrained to prevent liquid flow across the valve, and that can open in response to a pressure gradient. In various implementations, the fluid passages 226 may include fenestrations formed through the first film layer 204. Fenestrations may be formed by removing material from the first film layer 204, but the amount of material removed and the resulting dimensions of the fenestrations may be up to an order of magnitude less than perforations, and may not deform the edges.

In various implementations, the fluid passages 226 may include one or more slits, slots, or combinations of slits and slots in the first film layer 204. In various implementations, the fluid passages 226 may include linear slots having a length less than about five millimeters and a width less than about two millimeters. In various implementations, the length may be at least about two millimeters, and the width may be at least about 0.5 millimeters. In various implementations, the length may be in a range of about two millimeters to about five millimeters and the width may be in a range of about 0.5 millimeters to about two millimeters, with a tolerance of about 0.1 millimeters. In various implementations, the length may be about three millimeters. Such dimensions and tolerances may be achieved with a laser cutter, for example. In various implementations, slots of such configurations may function as imperfect valves that substantially reduce liquid flow in a normally closed or resting state. Such slots may form a flow restriction without being completely closed or sealed. The slots can expand or open wider in response to a pressure gradient applied across the slot to allow increased liquid flow through the slot.

In various implementations, the fluid passages 226 may include linear slits having a length of less than about five millimeters. In various implementations, the length of the linear slits may be at least about two millimeters. In various implementations, the length of the linear slits may be in a range of about two millimeters to about five millimeters, with a tolerance of about 0.1 millimeters. In various implementations, the length of the linear slits may be about three millimeters.

In various implementations, the manifold layer 206 may be formed as a substantially sheet-like structure having a top surface 230 opposite a bottom surface 232, and a periphery 234 defined by an outer perimeter of the manifold layer 206. In various implementations, the periphery 234 of the manifold layer 206 may be substantially similar to or coextensive with the periphery 224 of the first film layer 204. In various implementations, the manifold layer 206 may be formed from a sheet of polyurethane, such as a vacuum-formed sheet of polyurethane having a thickness of about 0.5 millimeters. In various implementations, the manifold layer 206 may be formed from a polymer material that is substantially clear or optically transparent, allowing the user to see through the manifold layer 206.

In various implementations, windows 236 may be removed from the manifold layer 206 and form a grid pattern. For example, the plurality of windows 236 may be arranged in a pattern of rows and columns. The center of each window 236 may be aligned with the center of each other window 236 within a row, and the center of each window 236 may be aligned with the center of each other window 236 within a column. In various implementations, a plurality of standoffs 238 may be formed on the bottom surface 232 of the manifold layer 206. In various implementations, the plurality of standoffs 238 may form a grid pattern. For example, the plurality of standoffs 238 may be arranged in a pattern of rows and columns. The center of each standoff 238 may be aligned with the center of each other standoff 238 within a row, and the center of each standoff 238 may be aligned with the center of each other standoff 238 within a column. In various implementations, each row of the plurality of windows 236 may be disposed adjacent to a row of the plurality of standoffs 238, and each column of the plurality of windows 236 may be disposed adjacent to a column of the plurality of standoffs 238. In various implementations, the plurality of windows 236 and the plurality of standoffs 238 may be arranged in a pattern such that rows of the pattern alternate between rows of the plurality of windows 236 and rows of the plurality of standoffs 238, and columns of the pattern alternate between columns of the plurality of windows 236 and columns of the plurality of standoffs 238.

In various implementations, each window 236 may be substantially circular in profile in the plane of the top surface 230 of the manifold layer 206. In various implementations, each standoff 238 may be substantially circular in profile and protrude outwardly in a substantially orthogonal manner from the plane of the bottom surface 232 of the manifold layer 206. In various implementations, a diameter of each window 236 may be greater than a diameter of each standoff 238. For example, each window 236 may have a diameter of about eight millimeters, and each standoff 238 may have a diameter of about three millimeters. In various implementations, each standoff 238 may have a height of in a range of about 0.5 millimeters to about 3 millimeters. In various implementations, each standoff 238 may have a height of about 2.5 millimeters. In various implementations, each standoff 238 may have a height of about 3 millimeters. In various implementations, each standoff 238 within a row may be spaced a distance of about four millimeters on center from an adjacent standoff 238 within a row, and each standoff 238 within a column may be spaced a distance of about four millimeters on center from an adjacent standoff 238 within a column. In various implementations, the plurality of standoffs 238 may be right cylinders with hemispherical ends, such as half-capsules, and may be formed on and protrude substantially away from the bottom surface 232 of the manifold layer 206 in a direction substantially normal to the bottom surface 232. In various implementations, each of the plurality of standoffs 238 may have a height in a range of about 2.5 millimeters to about three millimeters.

As shown in FIG. 2, examples of the manifold layer 206 may include a raised portion, such as a lip portion or a boss 240. In various implementations, the boss 240 may protrude outwardly in a substantially orthogonal manner from the plane of the top surface 230 of the manifold layer 206. In various implementations, the boss 240 may have a scaled down profile or outline similar to the shape of the periphery 234. In various implementations, the manifold layer 206 may also have a border region 242 between the boss 240 and the periphery 234, and the border region 242 may not include any windows 236 or standoffs 238. The manifold layer 206 may also include a fluid-ingress or instillation aperture, such as aperture 239, and a region surrounding the aperture 239 that does not have any windows 236 or standoffs 238, such as coupling region 243. In various implementations, the first film layer 204 may include a coupling region around the aperture 228, such as coupling region 241, for coupling the area around the aperture 228 to the coupling region 243. In various implementations, the coupling region 241 may include an area of the top surface 220 of the first film layer 204 surrounding the aperture 228. In various implementations, the coupling region 241 may be the region of the top surface 220 defined by an area between a circle having a diameter greater than the diameter of the aperture 228 and concentric with the aperture 228 and the aperture 228 itself. In various implementations, the coupling region 241 may be raised such that it is offset a distance away from the top surface 220 towards the manifold layer 206.

The second film layer 208 may have a top surface 244 opposite a bottom surface 246, and a periphery 248 defined by a perimeter of the second film layer 208. A negative-pressure aperture, such as aperture 250, and a fluid-ingress or instillation aperture, such as aperture 252, may be formed through the second film layer 208. In various implementations, the second film layer 208 may be formed from or include any of the materials previously described with respect to the cover 110 and/or the first film layer 204. In various implementations, the second film layer 208 may include a coupling region around the aperture 252, such as coupling region 253, for coupling the area around the aperture 252 to the coupling region 241. In various implementations, the coupling region 253 may include an area of the bottom surface 246 of the second film layer 208 surrounding the aperture 252. In various implementations, the coupling region 253 may be the region of the bottom surface 246 defined by an area between a circle having a diameter greater than the diameter of the aperture 252 (and concentric with the aperture 252) and the aperture 252 itself. In various implementations, the coupling region 253 may be raised such that it is offset a distance away from the bottom surface 246 towards the manifold layer 206.

As shown in FIG. 2, examples of the dressing 104 may include a cover 110 having a top surface 254 opposite a bottom surface 256, and a periphery 258 defined by an outer perimeter of the cover 110. A central aperture 260 may be formed through the cover 110.

In various implementations, the periphery 214 of the sealing layer 202 may be substantially coextensive with the periphery 258 of the cover 110. In various implementations, the periphery 224 of the first film layer 204, the periphery 234 of the manifold layer 206, and the periphery 248 of the second film layer 208 may be substantially coextensive. In various implementations, the outline of the treatment aperture 216 of the sealing layer 202 may be substantially coextensive with the outline of the central aperture 260 of the cover 110. In various implementations, the outlines of the treatment aperture 216 and the central aperture 260 may be substantially similar to the outlines of the periphery 224, periphery 234, and periphery 248. In various implementations, the outlines of the treatment aperture 216 and the central aperture 260 may be substantially similar to but scaled down from the outlines of the periphery 224, periphery 234, and periphery 248. In assembled form, the sealing layer 202, first film layer 204, manifold layer 206, second film layer 208, and cover 110 may be stacked such that the periphery 214 is aligned with the periphery 258, and the periphery 224 is aligned with the periphery 234 and periphery 248. In various implementations, the treatment aperture 216 may be aligned with the central aperture 260, and the periphery 224, periphery 234, and periphery 248 are positioned such that they are aligned with and evenly extend past the outlines of the treatment aperture 216 and the central aperture 260.

In various implementations, a portion of the top surface 220 of the first film layer 204 near the periphery 224 may be coupled to a portion of the bottom surface 232 of the manifold layer 206 at the border region 242, and a portion of the top surface 220 at the coupling region 241 may be coupled to a portion of the bottom surface 232 at the coupling region 243 to define a first chamber 245 between the first film layer 204 and the manifold layer 206. In various implementations, a portion of the bottom surface 246 of the second film layer 208 near the periphery 248 may be coupled to a portion of the top surface 230 of the manifold layer 206 at the border region 242, and a portion of the bottom surface 246 at the coupling region 253 may be coupled to a portion of the top surface 230 at the coupling region 243 to define a second chamber 247 between the second film layer 208 and the manifold layer 206. In various implementations, aperture 228 of the first film layer 204, aperture 239 of the manifold layer 206, and aperture 252 of the second film layer 208 may be substantially coaxially aligned. In various implementations, a fluid-ingress passage may be defined as the fluid passageway through the aperture 252, aperture 239, and aperture 228. In various implementations, as a result of the coupling regions 241 and 253 being coupled to the coupling region 243, the fluid-ingress passage may be substantially isolated from the first chamber 245 and the second chamber 247 formed in the interior of the dressing 104.

In various implementations, a portion of the top surface 210 of the sealing layer 202 around the treatment aperture 216 may be coupled to a portion of the bottom surface 222 of the first film layer 204 near the periphery 224, and a portion of the bottom surface 256 of the cover 110 around the central aperture 260 may be coupled to a portion of the top surface 244 of the second film layer 208 near the periphery 248. In various implementations, a portion of the top surface 210 of the sealing layer 202 between the periphery 214 and the treatment aperture 216 may be coupled to a portion of the bottom surface 256 of the cover 110 between the periphery 258 and the central aperture 260.

Some examples of the dressing 104 also include a dressing interface 262 and a fluid conductor 264. In various implementations, the fluid conductor 264 may be a flexible tube that can be fluidly coupled on one end to the dressing interface 262. In various implementations, the dressing interface 262 may be an elbow connector that can be placed over aperture 260 to provide a fluid path between the fluid conductor 264 and the interior of the dressing 104. In various implementations, the dressing 104 includes a fluid-ingress or instillation aperture cover, such as cover 266. In various implementations, the cover 266 may be formed from or include any of the materials previously described with respect to cover 110, first film layer 204, and/or second film layer 208. In various implementations, the cover 266 may be releasably coupled to a portion of the top surface 244 of the second film layer 208 and/or a portion of the top surface 254 of the cover 110 to cover the aperture 252, and may be removed by the user to expose the aperture 252 prior to the dressing 104 being used in instillation or oxygen therapy. For example, a bottom surface 267 of cover 266 may be coated with an adhesive having a release factor suitable to facilitate removal of the cover 266 by hand and without damaging or deforming the dressing 104.

As illustrated in FIG. 2, some examples of the dressing 104 may include a release liner 268 to protect the sealing layer 202 and the adhesive coated on the bottom surface 256 of the cover 110 prior to use. The release liner 268 may also provide stiffness to assist with, for example, deployment of the dressing 104. In various implementations, the release liner may include a polyethylene terephthalate (PET) or similar polar semi-crystalline polymer. The use of a polar semi-crystalline polymer for the release liner 268 may substantially preclude wrinkling or other deformation of the dressing 104. The polar semi-crystalline polymer may be highly orientated and resistant to softening, swelling, or other deformation that may occur when objects are brought into contact with the layers and/or components of the dressing 104, or when the dressing 104 is subjected to temperature or environmental variations, or during sterilization. Further, a release agent may be disposed on a top surface 270 of the release liner 268 that is configured to contact the bottom surface 212 of the sealing layer 202 and the adhesive disposed on the bottom surface 256 of the cover 110. For example, the release agent may be a silicone coating and may have a release factor suitable to facilitate removal of the release liner 268 by hand and without damaging or deforming the dressing 104. In various implementations, the release agent may be a fluorocarbon or a fluorosilicone. In various implementations, the release liner 268 may be uncoated or otherwise used without a release agent.

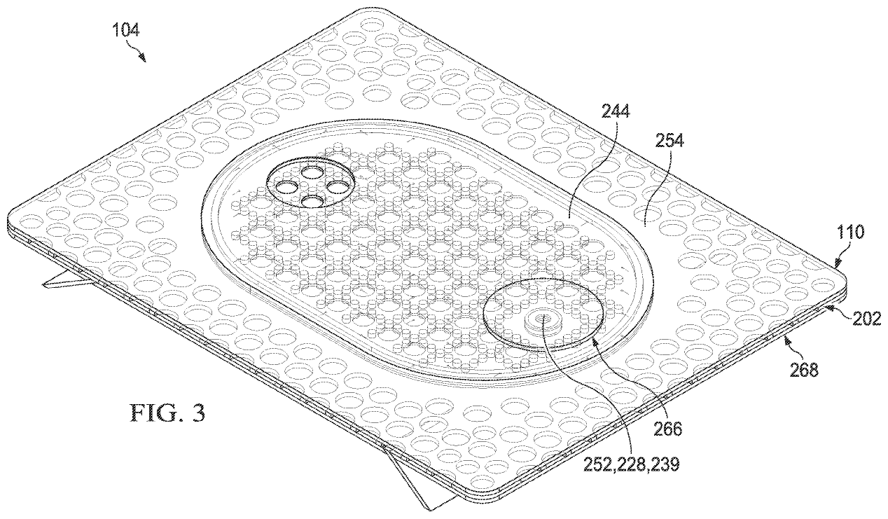

FIG. 3 is an isometric view of an assembled example of the dressing 104 of FIG. 2. As shown in FIG. 3, in some examples, the cover 110, the second film layer 208, the manifold layer 206, and/or the first film layer 204 may be substantially clear or optically transparent, allowing for visualization of the layers of the dressing 104 as well as visualization through the windows 236 of the manifold layer 206. In various implementations, the cover 266 may be coupled to the top surface 244 and/or the top surface 254, fluidly isolating the fluid pathway between the interior of the dressing 104 and an external environment via aperture 252. In such a configuration, the dressing 104 may be used in negative-pressure wound treatment without instillation or oxygen therapy.

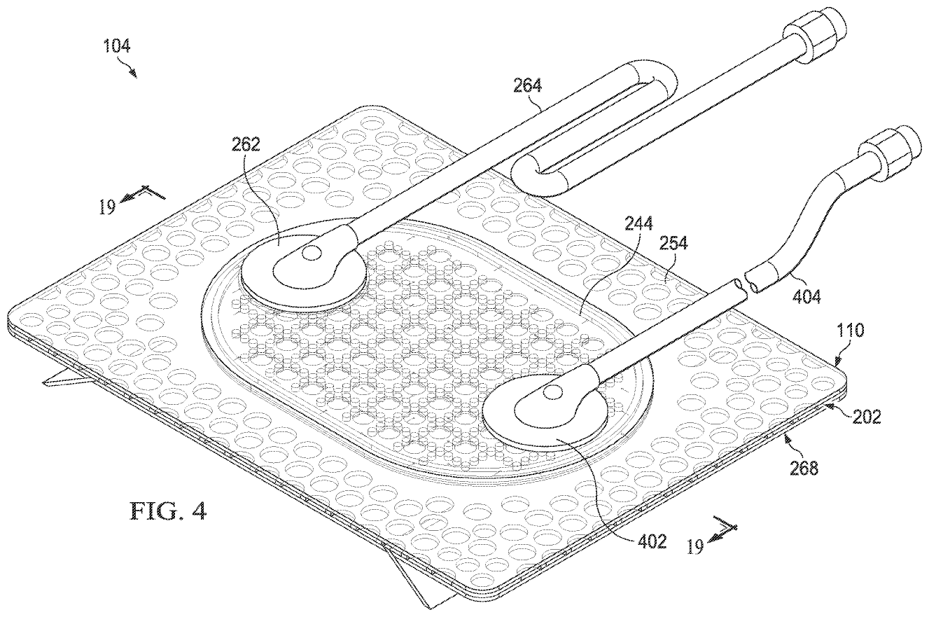

FIG. 4 is an isometric view of the assembled dressing 104 of FIGS. 2 and 3 with dressing interfaces for negative-pressure wound therapy and instillation and/or oxygen therapy attached. As shown in FIG. 3, in some examples, the dressing interface 262 and the fluid conductor 264 may be attached to the top surface 244 and/or top surface 254 and fluidly coupled to the interior of the dressing 104 via the aperture 250, and a dressing interface 402 and a fluid conductor 404 may be similarly attached to the top surface 244 and/or top surface 254 and fluidly coupled to the fluid-ingress passage via the aperture 252. In various implementations, the fluid conductor 264 may be connected to the negative-pressure source 102 and the fluid conductor 404 may be connected to the solution source 118 and/or an oxygen source.

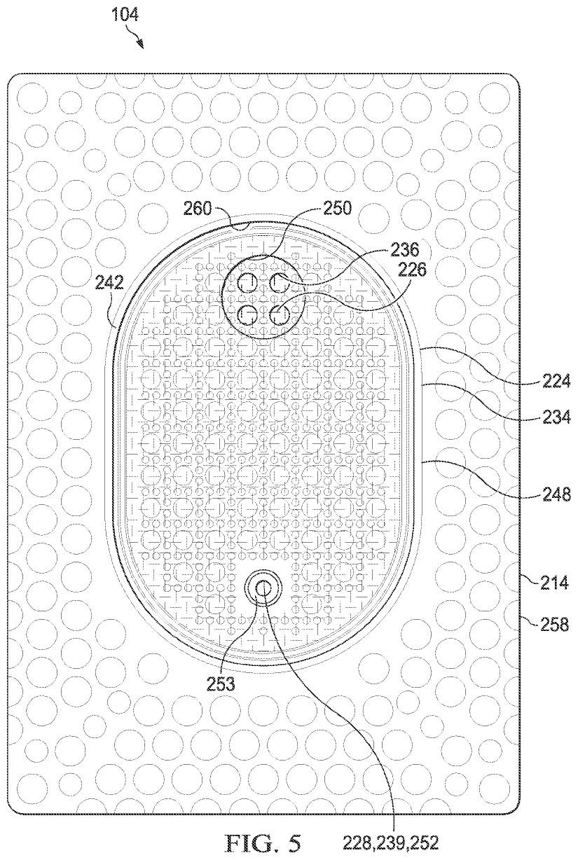

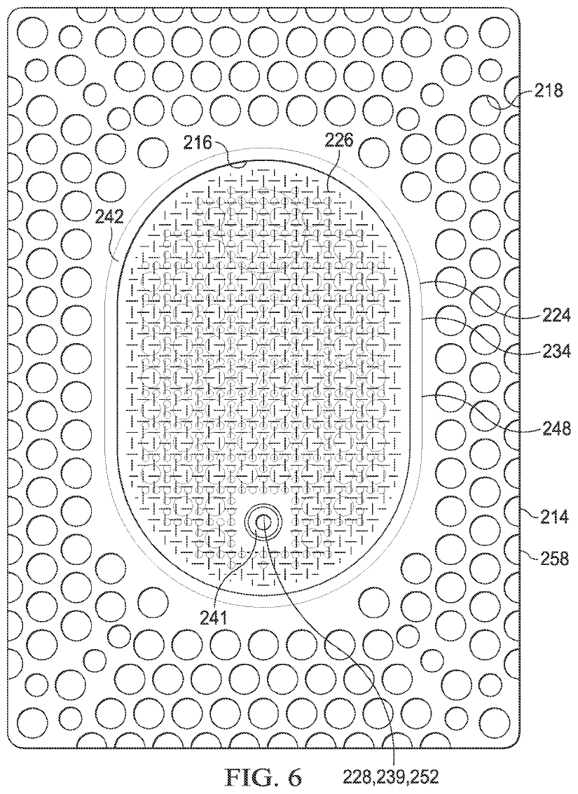

FIG. 5 is a top view of the dressing 104 of FIGS. 2-4, as assembled, illustrating details that may be associated with some examples. FIG. 6 is a bottom view of the dressing 104 of FIGS. 2-5, illustrating details that may be associated with some embodiments. As illustrated in FIGS. 5 and 6, in some examples of the dressing 104, the periphery 258 of the cover 110 may be coextensive with the periphery 214 of the sealing layer 202. In various implementations, the periphery 224 of the first film layer 204 may be coextensive with the periphery 234 of the manifold layer 206 and the periphery 248 of the second film layer 208. In various implementations, the perimeter or outline of the central aperture 260 of the cover 110 may be coextensive with the perimeter or outline of the treatment aperture 216 of the sealing layer 202 in a plane define by the top surface 254 of the cover 110 or the bottom surface 212 of the sealing layer 202.

As previously described with reference to FIG. 2, the perimeters or outlines of the treatment aperture 216 and the central aperture 260 may be similar in shape to the perimeters or outlines of the periphery 224, periphery 234, and periphery 248, but scaled down such that in assembled form, a portion of the sealing layer 202 surrounding the treatment aperture 216 overlaps with a portion of the first film layer 204 around the periphery 224, and a portion of the cover 110 surrounding the central apertures 260 overlaps with a portion of the second film layer 208 around the periphery 248—for example, at the border region 242. As shown in FIG. 5 and, in some examples, the outlines or perimeters of the aperture 252 of the second film layer 208, the aperture 239 of the manifold layer 206, and the aperture 228 of the first film layer 204 may be coextensive.

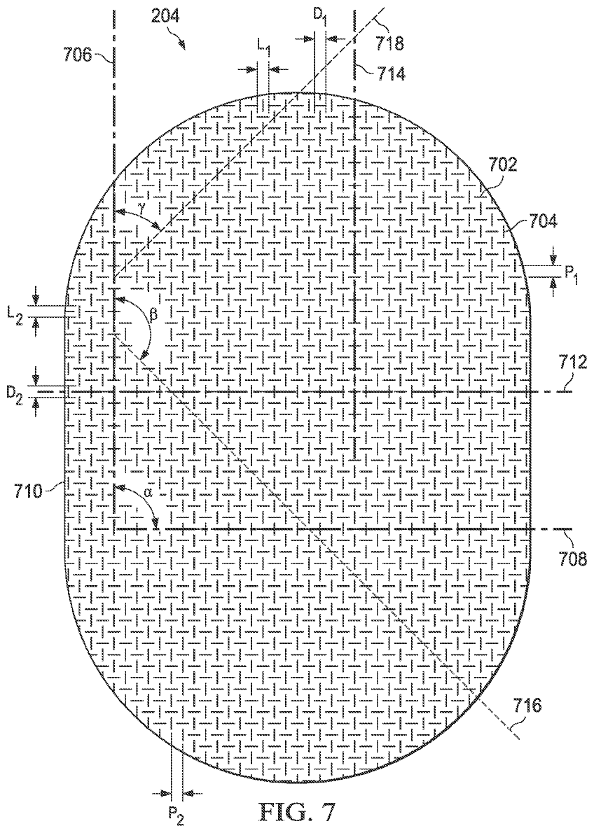

FIGS. 7-18 are top views illustrating additional details that may be associated with some examples of the first film layer 204. For example, as illustrated in FIG. 7, the fluid passages 226 may include a first plurality of perforations 702 and a second plurality of perforations 704. Each of the first plurality of perforations 702 and the second plurality of perforations 704 may be linear or curved perforations, such as slots or slits. In some embodiments where the perforations are linear slots or slits, each of the first plurality of perforations 702 may have a length L₁ and each of the second plurality of perforations 704 may have a length L₂. In some embodiments, where the perforations are curved slots or slits, each of the first plurality of perforations may have a length L₁ measured from an end of the curved slot or slit to the other end of the curved slot or slit, and each of the second plurality of perforations may have a length L₂ measured from an end of the curved slot or slit to the other end of the curved slot or slit. In some embodiments, the length L₁ may be equal to the length L₂. The first plurality of perforations 702 and the second plurality of perforations 704 may be distributed across the second layer in one or more rows in one direction or in different directions.

In example embodiments, each of the first plurality of perforations 702 may have a first long axis. In some embodiments, the first long axis may be parallel to a first reference line 706 running in a first direction. In illustrative examples, each of the second plurality of perforations 704 may have a second long axis. In example embodiments, the second long axis may be parallel to a second reference line 708 running in a second direction. In some embodiments, one or both of the first reference line 706 and the second reference line 708 may be defined relative to an edge 710 or line of symmetry of the first film layer 204. For example, one or both of the first reference line 706 and the second reference line 708 may be parallel or coincident with an edge 710 or line of symmetry of the first film layer 204. In some illustrative embodiments, one or both of the first reference line 706 and the second reference line 708 may be rotated an angle relative to an edge 710 of the first film layer 204. In example embodiments, an angle α may define the angle between the first reference line 706 and the second reference line 708.

In some example embodiments, the centroid of each of the first plurality of perforations 702 within a row may intersect a third reference line 712 running in a third direction. In illustrative embodiments, the centroid of each of the second plurality of perforations 704 within a row may intersect a fourth reference line 714 running in a fourth direction. In general, a centroid refers to the center of mass of a geometric object. In the case of a substantially two dimensional object such as a linear slit, the centroid of the linear slit will be the midpoint.

The pattern of fluid passages 226 may also be characterized by a pitch, which indicates the spacing between corresponding points on fluid passages 226 within a pattern. In example embodiments, pitch may indicate the spacing between the centroids of fluid passages 226 within the pattern. Some patterns may be characterized by a single pitch value, while others may be characterized by at least two pitch values. For example, if the spacing between centroids of the fluid passages 226 is the same in all orientations, the pitch may be characterized by a single value indicating the spacing between centroids in adjacent rows. In example embodiments, a pattern comprising a first plurality of perforations 702 and a second plurality of perforations 704 may be characterized by two pitch values, P₁ and P₂, where P₁ is the spacing between the centroids of each of the first plurality of perforations 702 in adjacent rows, and P₂ is the spacing between the centroids of each of the second plurality of perforations 704 in adjacent rows.

In example embodiments, within each row of the first plurality of perforations 702, each perforation may be separated from an adjacent perforation by a distance D₁. In some embodiments, within each row of the second plurality of perforations 704, each perforation may be separated from an adjacent perforation by a distance D₂. In some patterns, the rows may be staggered. The stagger may be characterized by an orientation of corresponding points in successive rows relative to an edge or other reference line associated with the first film layer 204. In some embodiments, the rows of the first plurality of perforations 702 may be staggered. For example, a fifth reference line 716 in a fifth direction runs through the centroids of corresponding perforations of adjacent rows of the first plurality of perforations 702. In some example embodiments, the stagger of the rows of the first plurality of perforations 702 may be characterized by the angle β formed between the first reference line 706 and the fifth reference line 716. In additional illustrative embodiments, the rows of the second plurality of perforations 704 may also be staggered. For example, a sixth reference line 718 in a sixth direction runs through the centroids of corresponding perforations of adjacent rows of the second plurality of perforations 704. In some embodiments, the stagger of the rows of the second plurality of perforations 704 may be characterized by the angle γ formed between the first reference line 706 and the sixth reference line 718.

FIG. 7 illustrates an example of a pattern that may be associated with some embodiments of the fluid passages 226. In the example of FIG. 7, each of the first plurality of perforations 702 and the second plurality of perforations 704 may be linear slots or slits. The first reference line 706 may be parallel with an edge 710, and the second reference line 708 may be orthogonal to the edge 710. In example embodiments, the third reference line 712 is orthogonal to the first reference line 706, and the fourth reference line 714 is orthogonal to the second reference line 708. For example, the third reference line 712 may be incident with the centroids of corresponding perforations in alternating rows of the second plurality of perforations 704, and the fourth reference line 714 may intersect the centroids of corresponding perforations in alternating rows of the first plurality of perforations 702. In the example of FIG. 7, the fluid passages 226 are arranged in a cross-pitch pattern in which each of the first plurality of perforations 702 is orthogonal along its first long axis to each of the second plurality of perforations 704 along its second long axis. For example, in FIG. 7, P₁ is equal to P₂ (within acceptable manufacturing tolerances), and the cross-pitch pattern may be characterized by a single pitch value. Additionally, L₁ and L₂ may be substantially equal, and D₁ and D₂ may also be substantially equal, all within acceptable manufacturing tolerances. The rows of the first plurality of perforations 702 and the rows of the second plurality of perforations 704 may be characterized as staggered. For example, in some example embodiments illustrated, α may be about 90°, β may be about 135°, γ may be about 45°, P₁ may be about 4 mm, P₂ may be about 4 mm, L₁ may be about 3 mm, L₂ may be about 3 mm, D₁ may be about 5 mm, and D₂ may be about 5 mm.

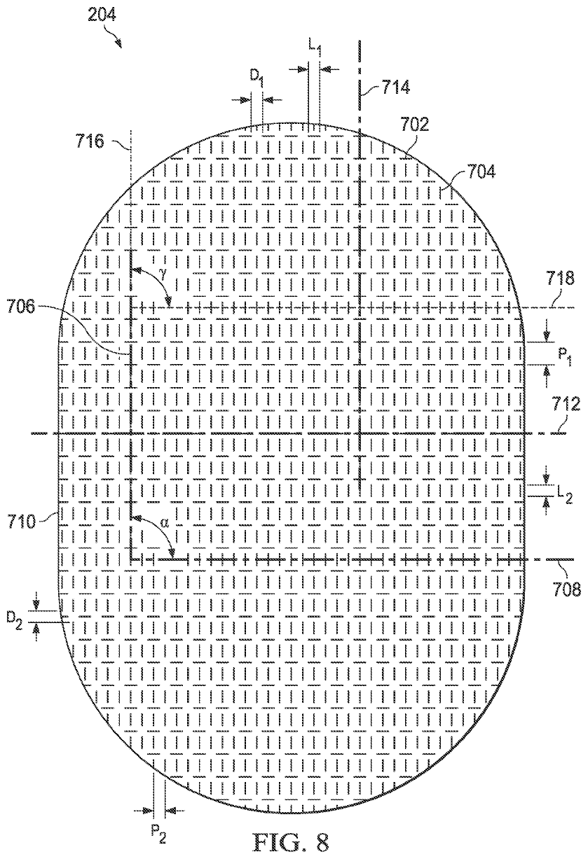

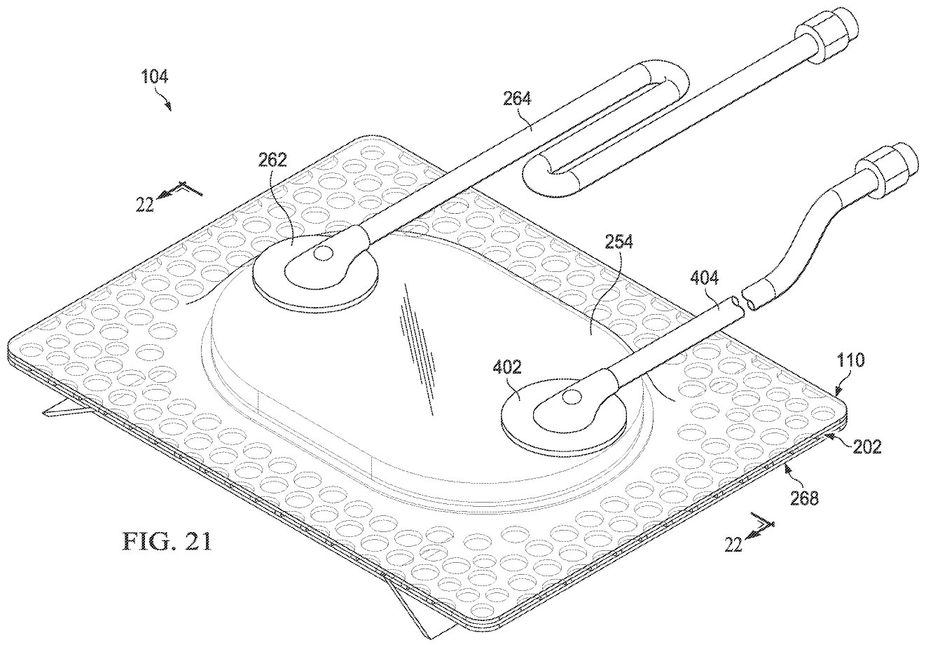

FIG. 8 is a schematic diagram of another example pattern that may be associated with some illustrative embodiments of the fluid passages 226. In illustrative examples of FIG. 21, each of the first plurality of perforations 702 and the second plurality of perforations 704 may be linear slits. The first reference line 706 may be parallel with the edge 710, and the second reference line 708 may be orthogonal to the edge 710. In some example embodiments, the third reference line 712 is orthogonal to the first reference line 706, and the fourth reference line 714 is orthogonal to the second reference line 708. In the example of FIG. 21, the third reference line 712 does not intersect or touch any of the second plurality of perforations 704, and the fourth reference line 714 may intersect the centroids of corresponding perforations in alternating rows of the first plurality of perforations 702. In example embodiments, the third reference line 712 may be equidistant from the centroids of corresponding adjacent perforations within each row of the second plurality of perforations 704. The pattern of FIG. 21 may also be characterized as a cross-pitch pattern, in which P₁ is not equal to P₂. In the example of FIG. 21, P₁ is larger than P₂. Additionally, L₁, L₂, D₁, and D₂ are substantially equal in the example of FIG. 21. In some embodiments, α may be about 90°, β may be about 0° such that the first reference line 706 is coincident with the fifth reference line 740, γ may be about 90°, P₁ may be about 6 mm, P₂ may be about 3 mm, L₁ may be about 3 mm, L₂ may be about 3 mm, D₁ may be about 3 mm, and D₂ may be about 3 mm.

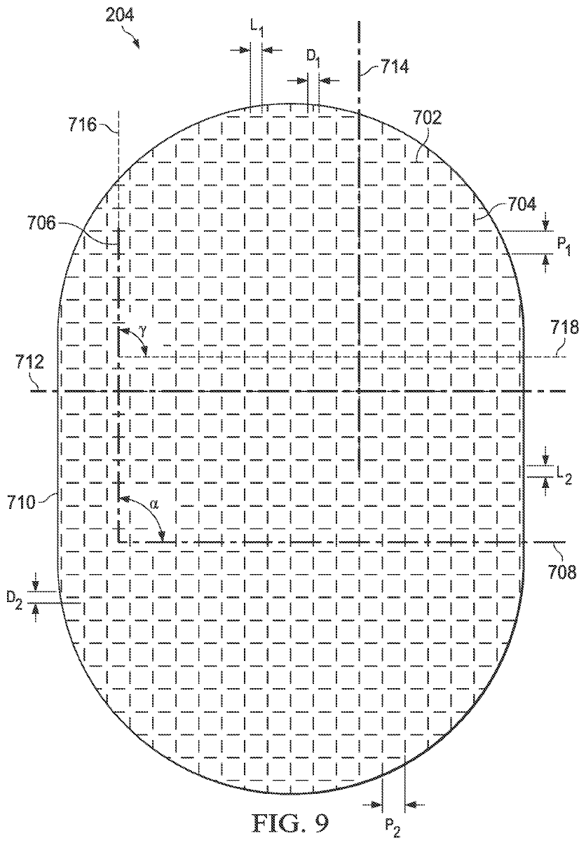

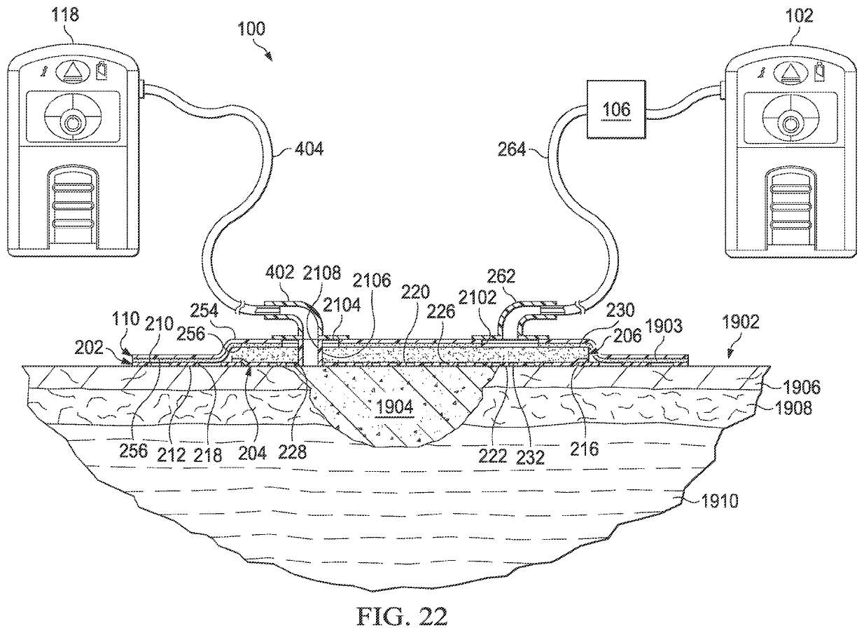

FIG. 9 illustrates an additional example of a pattern that can be associated with some embodiments of the fluid passages 226. In the example of FIG. 9, each of the first plurality of perforations 702 and the second plurality of perforations 704 may be linear slits. The first reference line 706 may be parallel with an edge 710, and the second reference line 708 may be orthogonal to an edge 710. In example embodiments, the third reference line 712 is orthogonal to the first reference line 706, and the fourth reference line 714 is orthogonal to the second reference line 708. In the example of FIG. 22, the third reference line 712 does not intersect or touch any of the second plurality of perforations 704, and the fourth reference line 714 does not intersect or touch any of the first plurality of perforations 702. In example embodiments, the third reference line 712 may be equidistant from the centroids of corresponding adjacent perforations within each row of the second plurality of perforations 704, and the fourth reference line 714 may be equidistant from the centroids of corresponding adjacent perorations within each row of the first plurality of perforations 702. The pattern of FIG. 22 may be characterized as a cross-pitch pattern, in which P₁ is substantially equal to P₂. Additionally, L₁, L₂, D₁, and D₂ are substantially equal in the example of FIG. 9. In some embodiments, α may be about 90°, β may be about 0° such that the first reference line 706 is coincident with the fifth reference line 740, γ may be about 90°, P₁ may be about 6 mm, P₂ may be about 6 mm, L₁ may be about 3 mm, L₂ may be about 3 mm, D₁ may be about 3 mm, and D₂ may be about 3 mm.

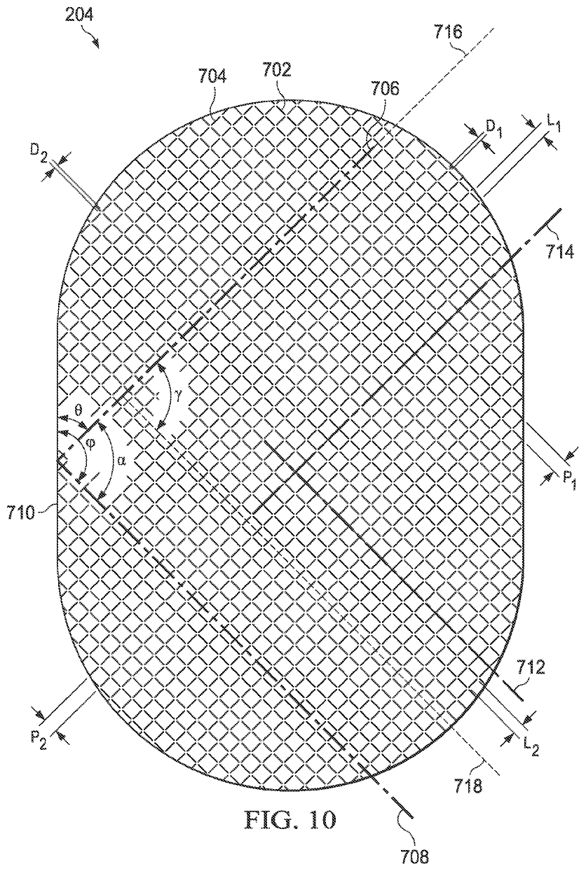

FIG. 10 illustrates additional embodiments of a pattern that may be associated with some embodiments of the fluid passages 226. In the example of FIG. 10, each of the first plurality of perforations 702 and the second plurality of perforations 704 may be linear slits. The first reference line 706 may form an angle θ with an edge 710, and the second reference line 708 may form an angle φ an edge 710. In example embodiments, the third reference line 712 is orthogonal to the first reference line 706, and the fourth reference line 714 is orthogonal to the second reference line 708. In the example of FIG. 10, the third reference line 712 does not intersect or touch any of the second plurality of perforations 704, and the fourth reference line 714 does not intersect or touch any of the first plurality of perforations 702. In example embodiments, the third reference line 712 may be equidistant from the centroids of corresponding adjacent perforations within each row of the second plurality of perforations 704, and the fourth reference line 714 may be equidistant from the centroids of corresponding adjacent perorations within each row of the first plurality of perforations 702. The pattern of FIG. 10 may be characterized as a cross-pitch pattern, in which P₁ is substantially equal to P₂. Additionally, L₁ may be substantially equal to L₂, and D₁ may be substantially equal to D₂ in the example of FIG. 10. In some embodiments, β may be about 0° such that the first reference line 706 is coincident with the fifth reference line 716, γ may be about 90°, θ may be about 45°, and φ may be about 135°.

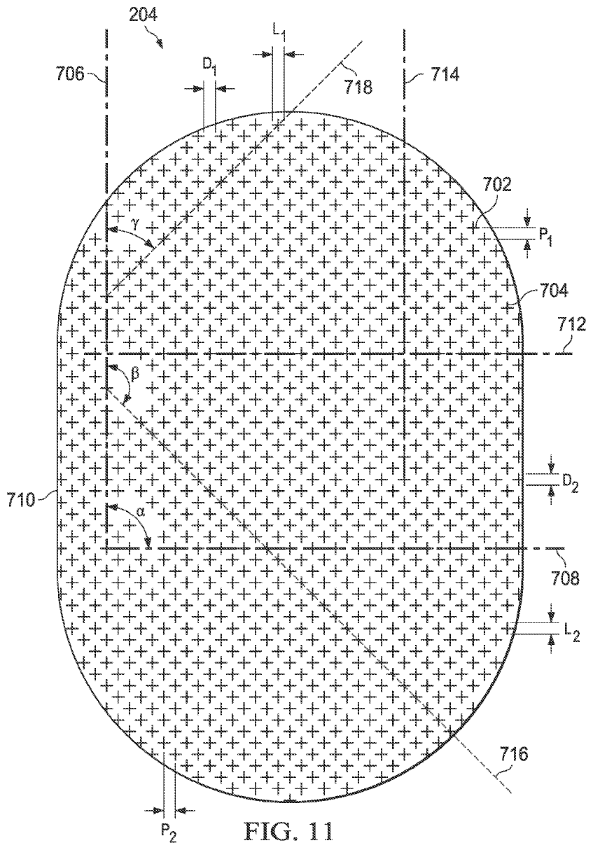

FIG. 11 illustrates examples that may be associated with some embodiments of the fluid passages 226. In some embodiments of FIG. 11, each of the first plurality of perforations 702 and the second plurality of perforations 704 may be linear slits. The first reference line 706 may be parallel with an edge 710, and the second reference line 708 may be orthogonal to an edge 710. In example embodiments, the third reference line 712 is orthogonal to the first reference line 706, and the fourth reference line 714 is orthogonal to the second reference line 708. For example, the third reference line 712 may be incident with the centroids of corresponding perforations in alternating rows of the second plurality of perforations 704, and the fourth reference line 714 may be incident with the centroids of corresponding perforations in alternating rows of the first plurality of perforations 702. In the example of FIG. 11, the centroid of each perforation of the first plurality of perforations 702 is incident with the centroid of a perforation of the second plurality of perforations 704. The fluid passages 226 are arranged in a cross-pitch pattern in which each of the first plurality of perforations 702 is orthogonal along its first long axis to each of the second plurality of perforations 704 along its second long axis. For example, in FIG. 11, P₁ is substantially equal to P₂, and the cross-pitch pattern may be characterized by a single pitch value. Additionally, L₁ and L₂ may be substantially equal, and D₁ and D₂ may also be substantially equal, all within acceptable manufacturing tolerances. The rows of the first plurality of perforations 702 and the rows of the second plurality of perforations 704 may be characterized as staggered. In some example embodiments of FIG. 11, α may be about 90°, β may be about 135°, γ may be about 45°.

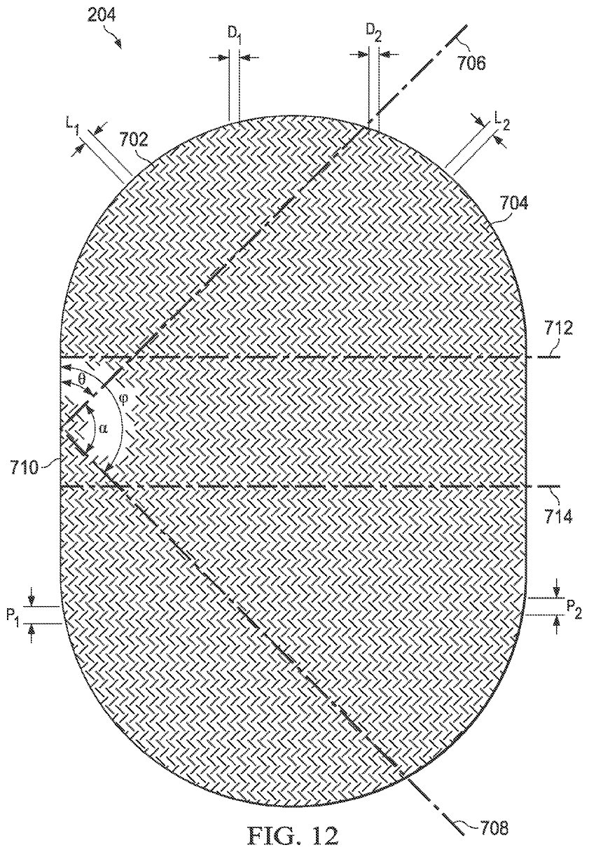

FIG. 12 show additional embodiments associated with certain illustrative embodiments of the fluid passages 226. In the example of FIG. 12, each of the first plurality of perforations 702 and the second plurality of perforations 704 may be linear slits. The first reference line 706 may form an angle θ with an edge 710. The second reference line 708 may form an angle φ with an edge 710. In example embodiments of FIG. 12, the third reference line 712 and the fourth reference line 714 may be orthogonal to an edge 710. In the example of FIG. 12, the rows of the first plurality of perforations 702 and the rows of the second plurality of perforations 704 may be characterized as mirrored rows running in one direction parallel with an edge 710 of the first film layer 204. For example, L₁ and L₂ may be substantially equal, D₁ and D₂ may be substantially equal, and P₁ and P₂ may be substantially equal, within acceptable manufacturing tolerances. In some embodiments, θ may be about 45°, and φ may be about 135°. The pattern of FIG. 12 may be characterized as a herringbone pattern.

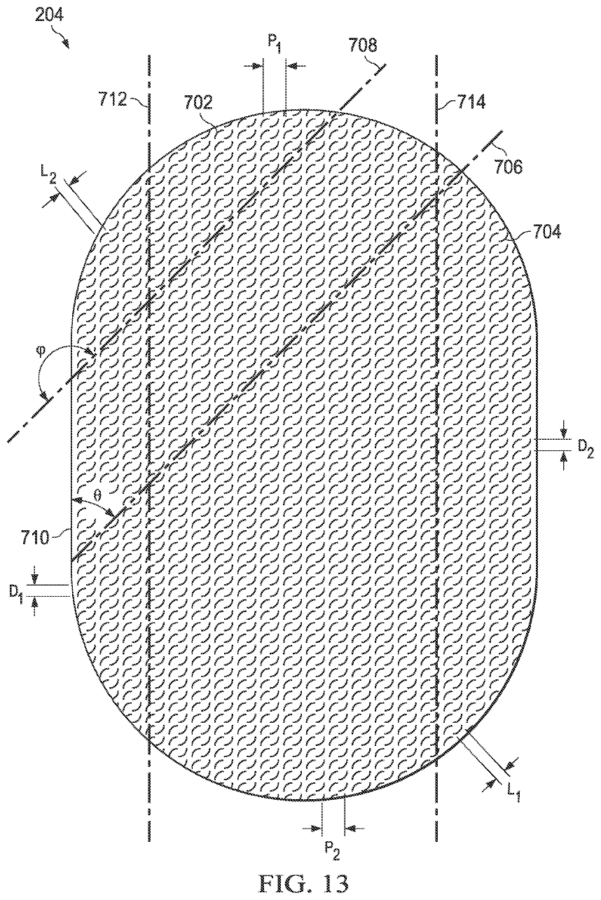

FIG. 13 shows additional example embodiments associated with certain illustrative embodiments of the fluid passages 226. In the example of FIG. 13, each of the first plurality of perforations 702 and the second plurality of perforations 704 may be curved slits. The first reference line 706 may form an angle θ with an edge 710. The second reference line 708 may form an angle φ with an edge 710. In example embodiments of FIG. 13, the third reference line 712 and the fourth reference line 714 may be parallel to an edge 710. In the example of FIG. 13, the rows of the first plurality of perforations 702 and the rows of the second plurality of perforations 704 may be characterized as mirrored rows running in one direction parallel with an edge 710 of the first film layer 204. The rows of the first plurality of perforations 702 and the rows of the second plurality of perforations 704 may be characterized as in an embodiment of FIG. 13. For example, L₁ and L₂ may be substantially equal, D₁ and D₂ may be substantially equal, and P₁ and P₂ may be substantially equal, within acceptable manufacturing tolerances. In some embodiments, θ may be about 45°, and φ may be about 225°.

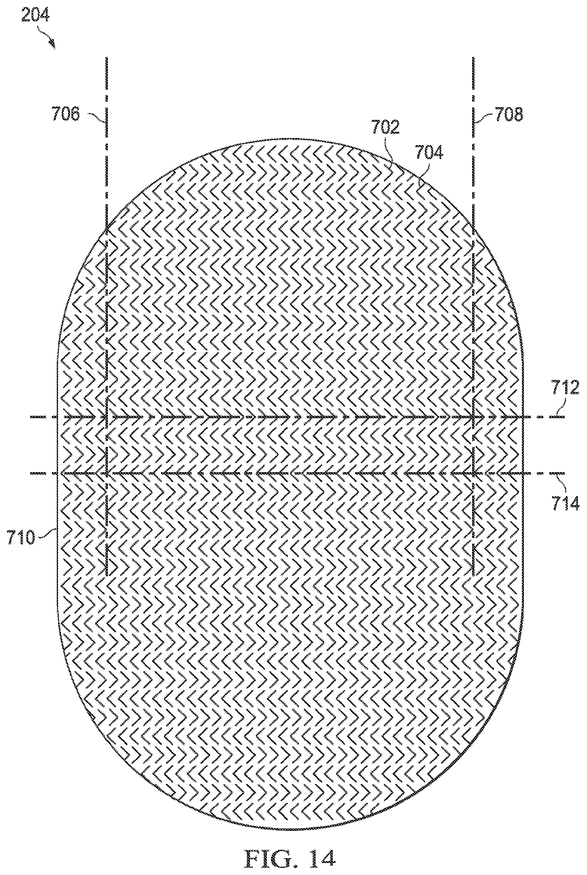

FIG. 14 shows additional embodiments associated with certain embodiments of the fluid passages 226. In the example of FIG. 14, each of the first plurality of perforations 702 and the second plurality of perforations 704 may be characterized as chevron slits. Each chevron slit may be formed from two orthogonal linear slits of the same length coincident at an endpoint. The chevron slit may be characterized as pointing in the direction defined by the vector drawn from the centroid of the chevron slit to the coincident endpoints. Within each row of the first plurality of perforations 702, the chevron slits point in the same direction. Within each row of the second plurality of perforations 704, the chevron slits point in the same direction. In example embodiments, the chevron slits of the first plurality of perforations 702 and the chevron slits of the second plurality of perforations 704 point in opposite directions. In example embodiments, the first reference line 706 and the second reference line 708 may be parallel with an edge 710. In illustrative embodiments, the third reference line 712 and the fourth reference line 714 may be orthogonal to the first reference line 706. In the example of FIG. 14, the rows of the first plurality of perforations 702 and the rows of the second plurality of perforations 704 may be characterized as mirrored rows running in one direction orthogonal to an edge 710 of the first film layer 204.

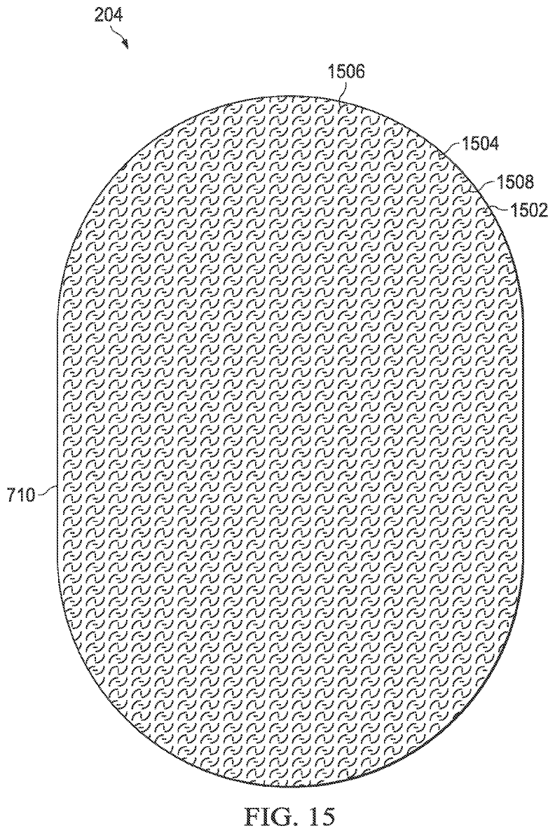

FIG. 15 further illustrates example embodiments that may be associated with some embodiments of the fluid passages 226. Certain patterns of the fluid passages 226 may comprise a third plurality of perforations 1502, a fourth plurality of perforations 1504, a fifth plurality of perforations 1506, and a sixth plurality of perforations 1508. Each of the third plurality of perforations 1502 may be a linear slit substantially orthogonal along a long axis to the edge 710. Each of the fourth plurality of perforations 1504 may be a linear slit substantially orthogonal to the long axis of third plurality of perforations 1502 along a long axis. Each of the fifth plurality of perforations 1506 may be a curved slit with its long axis rotated to form a 45° angle with the edge 710. Each of the sixth plurality of perforations 1508 may be a curved slit with its long axis rotated to form a 225° angle with the edge 710. Within each row, the pattern of fluid passages 226 may be a repeating pattern of one of the fifth plurality of perforations 1506, one of the third plurality of perforations 1502, one of the sixth plurality of perforations, 2820, one of the fifth plurality of perforations 1506, one of the third plurality of perforations 1502, and one of the sixth plurality of perforations 1508, in sequence. Each alternating row of the pattern of fluid passages 226 may be shifted three positions, in either direction.

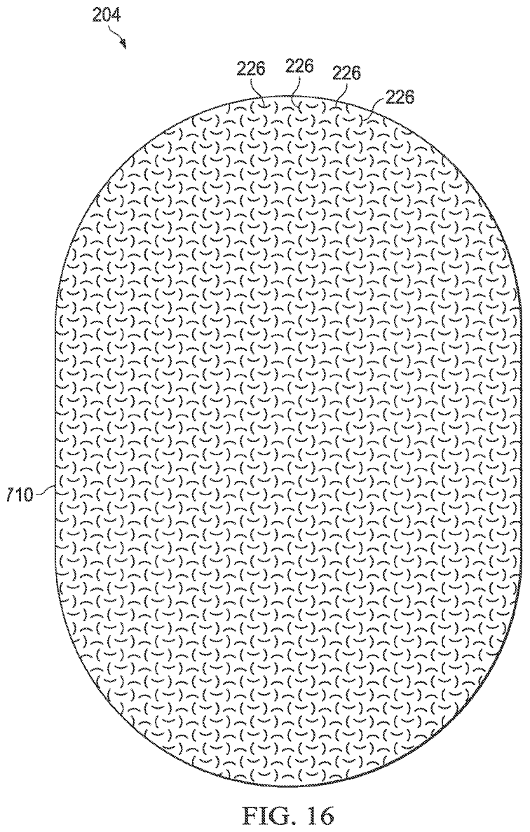

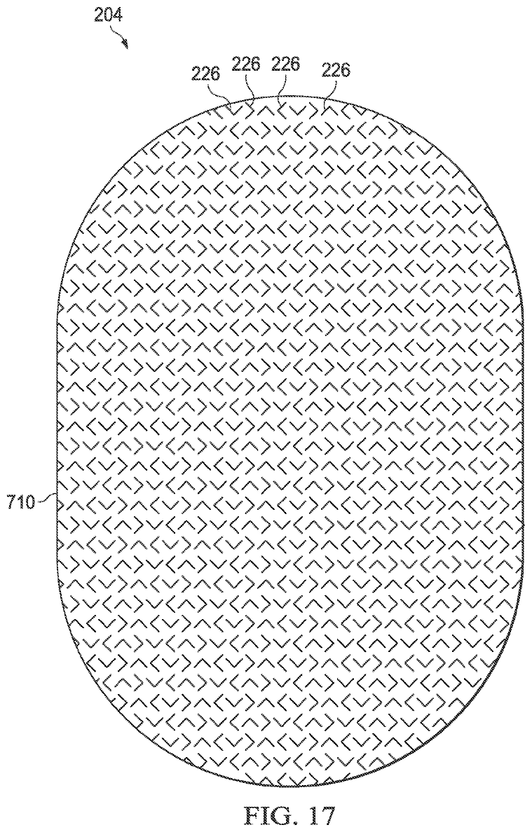

FIGS. 16 through 18 are schematic diagrams illustrating additional details that may be associated with some embodiments of the fluid passages 226. For example, as illustrated in FIG. 17, the fluid passages 226 may be distributed across the first film layer 204 in a pattern of rows. In some embodiments, each fluid passage 226 along a row may be rotated about 90° with respect to an adjacent fluid passage 226. Each fluid passage 226 along a row may be rotated about 90° clockwise or 90° counterclockwise with respect to a preceding adjacent fluid passage 226 in the row. In example embodiments of the pattern of fluid passages 226, every second row may be offset by one fluid passage 226 with respect to the previous row. The pattern of FIGS. 16 through 18 may be characterized as a pattern of offset rows. Example embodiments of the pattern of FIGS. 16 through 18 may additionally be characterized as a pattern of rotating fluid passages 22.

FIG. 16 illustrates example embodiments where the fluid passages 226 comprise curved slits. In some example embodiments, the fluid passages 226 within a row alternate between being parallel with the edge 710 of the first film layer 204 along a long axis of the fluid passage 226 and being orthogonal to the edge 710 of the first film layer 204 along the long axis.

FIG. 17 shows some embodiments where the fluid passages 226 comprise chevron slits. In some example embodiments, the fluid passages 226 within a row alternate between being parallel with the edge 710 of the first film layer 204 along a long axis of the fluid passage 226 and being orthogonal to the edge 710 of the first film layer 204 along the long axis.

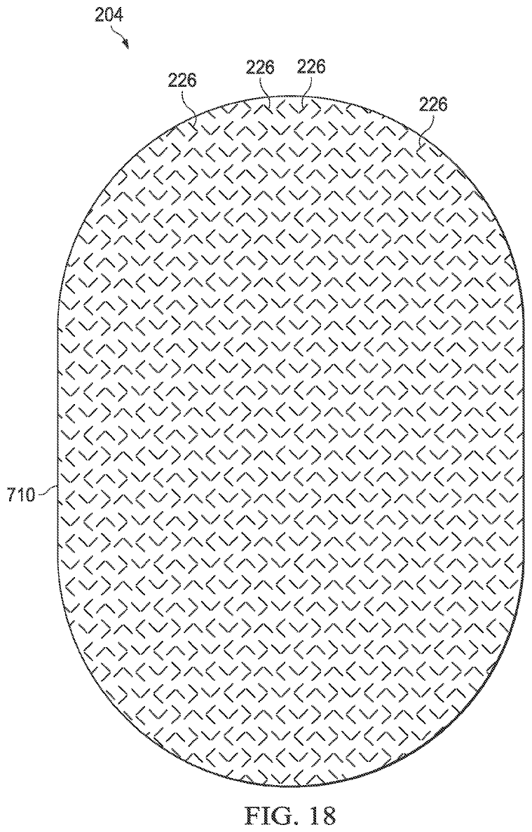

FIG. 18 further depicts illustrative embodiments where the fluid passages 226 comprise split-chevron slits. Each split-chevron slit may be formed from two orthogonal non-incidental linear slits mirrored about an axis bisecting the angle formed by the intersection of the orthogonal long axis of the linear slits. In some example embodiments, the fluid passages 226 within a row alternate between being parallel with the edge 710 of the first film layer 204 along a long axis of the fluid passage 226 and being orthogonal to the edge 710 of the second layer along the long axis.

In additional embodiments, P₁ may be in a range of about 4 millimeters to about 6 millimeters, P₂ may be in a range of about 3 mm to about 6 mm. In illustrative embodiments, D₁ may be in a range of about 3 mm to about 5 mm, and D₂ may be in a range of about 3 mm to 5 mm. In some embodiments, there may be an equal number of fluid passages 226 in the first plurality of perforations 702 as the number of fluid passages 226 in the second plurality of perforations 704.

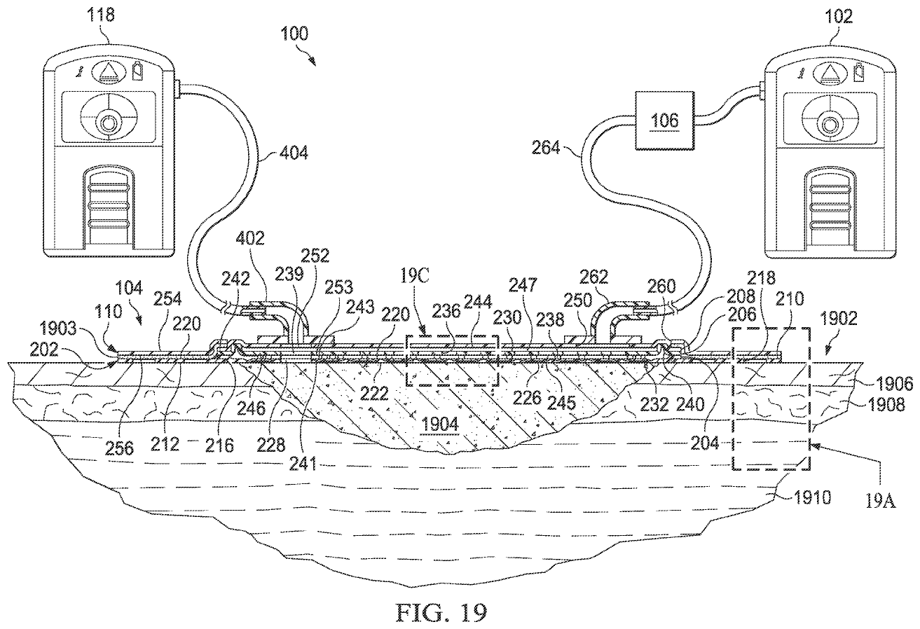

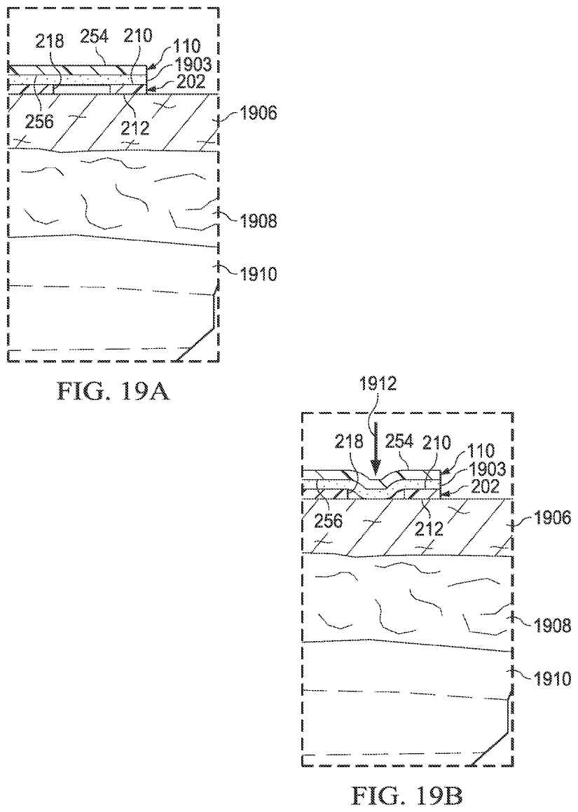

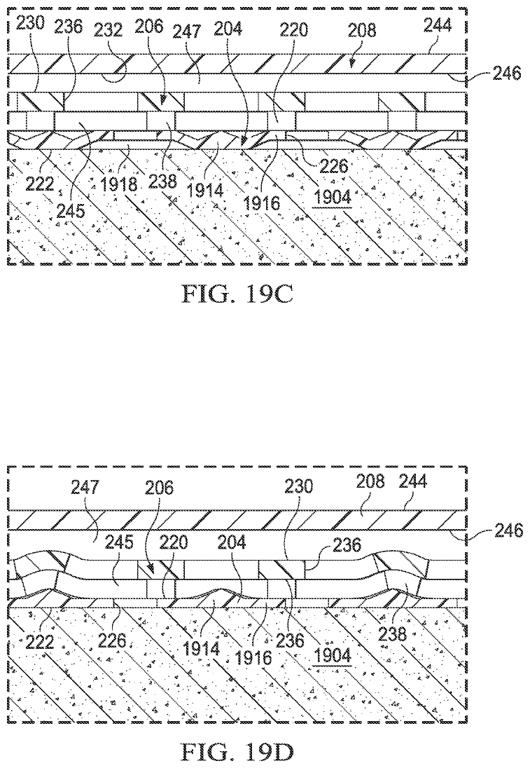

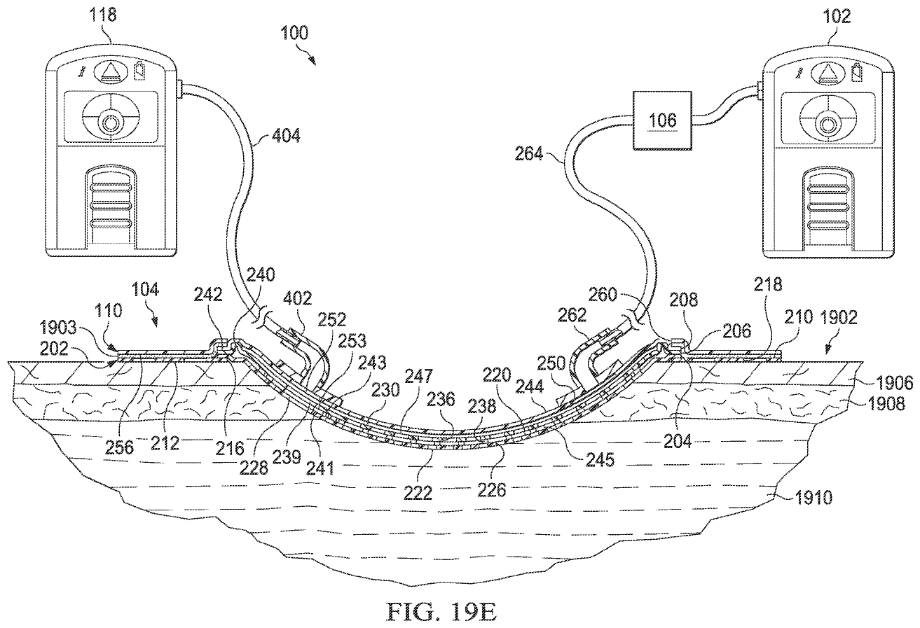

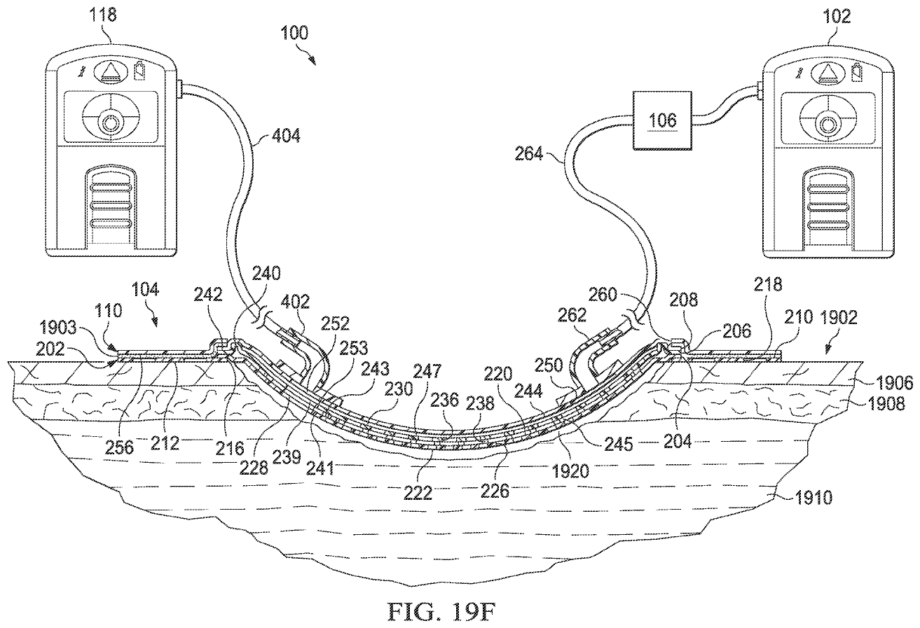

FIG. 19 is a cross-sectional view of the example dressing 104 of FIG. 4, taken at line 19-19, applied to an example tissue site 1902, and illustrating additional details associated with the therapy system 100 of FIG. 1. As shown in FIG. 19, in various implementations, the bottom surface 256 of the cover 110 may be coated with an adhesive layer 1903, and at least a portion of the cover 110 may be coupled to at least a portion of the top surface 220 of the sealing layer 202 with the adhesive layer 1903. The adhesive layer 1903 may be any of the attachment devices previously discussed with reference to FIG. 1. At least a portion of the bottom surface 256 of the cover 110 may be coupled to at least a portion of the top surface 244 of the second film layer 208, for example, at the border region 242, by the adhesive layer 1903. In various implementations, at least a portion of the bottom surface 222 of the first film layer 204—for example, at the border region 242—may be coupled to at least a portion of the top surface 210 of the sealing layer 202. In various implementations, the perimeters or outlines of the aperture 228, aperture 239, and aperture 252 may be aligned, and a portion of the first film layer 204 around the aperture 228 may be coupled to the coupling region 241 of the manifold layer 206, and a portion of the second film layer 208 around the aperture 252 may be coupled to the coupling region 241 to define a fluid-ingress passage through the aperture 228, aperture 239, and aperture 252. In various implementations, the fluid-ingress passage is substantially isolated from the interior of the dressing 104.

In some applications, the dressing 104 may be applied to a tissue site 1902 and cover a wound 1904. The tissue site 1902 may be or may include a defect or targeted treatment site, such as the wound 1904, which may be partially or completely filled or covered by the dressing 104. In some examples, the wound 1904 may be in the epidermis 1906. In some examples, the wound 1904 may extend through the epidermis 1906 and into a dermis 1908. In some examples, as shown in FIG. 19, the wound 1904 may extend through the epidermis 1906 and dermis 1908 into a subcutaneous tissue 1910. In some applications, at least a portion of the bottom surface 212 of the sealing layer 202 may be brought into contact with a portion of the epidermis 1906 surrounding the wound 1904, and at least a portion of the bottom surface 222 of the first film layer 204 may be placed within, over, on, against, or otherwise proximate to the wound 1904.

In operation, negative pressure may be provided to the wound 1904, and/or fluid may be removed from the wound 1904 by the negative-pressure source 102. For example, fluid may travel from the wound 1904 through at least one of the fluid passages 226 into the first chamber 245 between the top surface 220 of the first film layer 204, the bottom surface 232 of the manifold layer 206, and the standoffs 238. The fluid may then travel through the windows 236 of the manifold layer 206 and into the second chamber 247 between the top surface 230 of the manifold layer 206 and the bottom surface 246 of the second film layer 208. The fluid may then travel through the aperture 250 and into the dressing interface 262, and from the dressing interface 262 to the negative-pressure source 102 through the fluid conductor 264 and/or the container 106.

In operation, instillation solution may be provided to the wound 1904 by the solution source 118, and/or oxygen may be provided to the wound by an oxygen source. For example, instillation solution and/or oxygen may travel from the solution source 118 and/or the oxygen source to the dressing interface 402 via the fluid conductor 404. The instillation solution and/or oxygen may then travel from the dressing interface 402 to the dressing via the fluid-ingress passage formed by the aperture 252 of the second film layer 208, the aperture 239 of the manifold layer 206, and the aperture 228 of the first film layer 204. In such a configuration, the instillation solution and/or oxygen bypasses the interior spaces of the dressing 104 (such as the first chamber 245 and the second chamber 247) by traveling directly to the wound 1904 through the fluid-ingress passage, which may be substantially isolated from the interior spaces of the dressing 104.

FIG. 19A is a detail view, taken at reference 19A in FIG. 19, illustrating details that may be associated with some example embodiments of the example dressing 104 of FIG. 19. In various implementations, the sealing layer 202 may be sufficiently tacky at the bottom surface 212 to hold the dressing 104 in position relative to the epidermis 1906 and/or the wound 1904, while allowing the dressing 104 to be removed or repositioned without trauma to the tissue site 1902. In various implementations, the sealing layer 202 may be formed of a silicone polyurethane material, which may form sealing couplings at the bottom surface 212 with the epidermis 1906. In various implementations, the bond strength or tackiness of the sealing couplings may have a peel adhesion or resistance to being peeled form a stainless steel material between about 0.5 N/25 mm to about 1.5 N/25 mm on stainless steel substrate at about 25° C. at about 50% relative humidity based on ASTM D3330. The sealing layer 202 may achieve this bond strength after a contact time of less than about 60 seconds. Tackiness may be considered a bond strength of an adhesive after a very low contact time between the adhesive and a substrate. In various implementations, the sealing layer 202 may have a thickness in a range of about 200 micrometers to about 1,000 micrometers. Removing the release liner 268 may also expose adhesive layer 1903 through the apertures 218 of the sealing layer 202. In the assembled state, the thickness of the sealing layer 202 may create a gap between the adhesive layer 1903 and the epidermis 1906 through the apertures 218 of the sealing layer 202 such that the adhesive 1903 is not in contact with the epidermis 1906.

FIG. 19B is a detail view illustrating additional details that may be associated with the detail view of FIG. 19A in some implementations of the dressing 104 of FIG. 19. FIG. 19B illustrates the adhesive layer 1903 after a portion of it has been brought into contact with the epidermis 1906 by a force vector 1912 applied to the top surface 254 of the cover 110 at the apertures 218. In use, if the assembled dressing 104 is at the desired location, the force vector 1912 may be applied to the top surface 254 at the apertures 218 to cause at least a portion of the adhesive layer 1903 to be pressed at least partially into contact with the epidermis 1906 through at least one or more of the apertures 218 to form bonding couplings. The bonding couplings may provide secure, releasable mechanical fixation of the dressing 104 to the epidermis 1906. In various implementations, the sealing couplings may not be as mechanically strong as the bonding couplings. The bonding couplings may anchor the dressing 104 to the epidermis 1906, inhibiting and/or substantially preventing migration of the dressing 104.

FIG. 19C is a detail view, taken at reference 19C in FIG. 19, illustrating features that may be associated with some embodiments of the example dressing 104 of FIG. 19. As shown in FIG. 19C, in some examples, the first film layer 204 may include a plurality of micro features to ensure that the bottom surface 222 of the first film layer 204 is smooth when negative-pressure is applied to the dressing 104, but patterned in the absence of negative pressure. For example, the first film layer 204 may include a plurality of thickened regions 1914, and a plurality of thinner regions 1916 surrounding the thickened regions 1914. As illustrated in FIG. 19C, in the absence of negative pressure, the thin regions 1916 may be shaped so as to bias portions of the bottom surface 222 of the first film layer 204 away from the wound 1904 to define a plurality of voids 1918. In various implementations, these voids 1918 may provide additional spaces for instillation solution and/or oxygen to flow into in order to provide for increased saturation of the wound 1904 during instillation and/or oxygen therapy.

FIG. 19D is a detail view illustrating additional features, which may be associated with the detail view of FIG. 19C in some implementations of the dressing 104 of FIG. 19. As illustrated in FIG. 19D, in various implementations, the thinner regions 1916 may deform after negative pressure is applied to the dressing 104 such that the voids 1918 disappear and the bottom surface 222 lies substantially flat against the surface of the wound 1904, minimizing surface stresses against the wound 1904 by evenly spreading the pressure of the bottom surface 222 along a greater surface area of the wound 1904.

FIGS. 19E and 19F are cross-sectional views illustrating additional details, which may be associated with the dressing 104 of FIG. 19. FIGS. 19E and 19F illustrate examples of the dressing 104 applied to a tissue site 1902 having a contour. In the example of FIG. 19E, negative pressure has been applied to the dressing 104. Due to the pressure differential created between negative pressure in the dressing and the external ambient environment, force is applied to the top surface 244 of the second film layer 208 by the atmosphere, and the dressing 104 is biased towards the tissue site 1902 such that the bottom surface 222 of the first film layer 204 may be substantially in contact with the contours of the wound site 1902. FIG. 19F illustrates examples of the dressing 104 after negative pressure has been released from the dressing 104. In various implementations, after negative pressure is released, the dressing 104 my rebound away from the tissue site 1902 such that a void 1920 is created between the bottom surface 222 of the first film layer 204 and the tissue site 1902. In various implementations, the presence of the void 1920 may be beneficial during instillation therapy, as it creates additional volume between the bottom surface 222 and the surface of the tissue site 1902 for the instillation solution and/or oxygen to saturate.

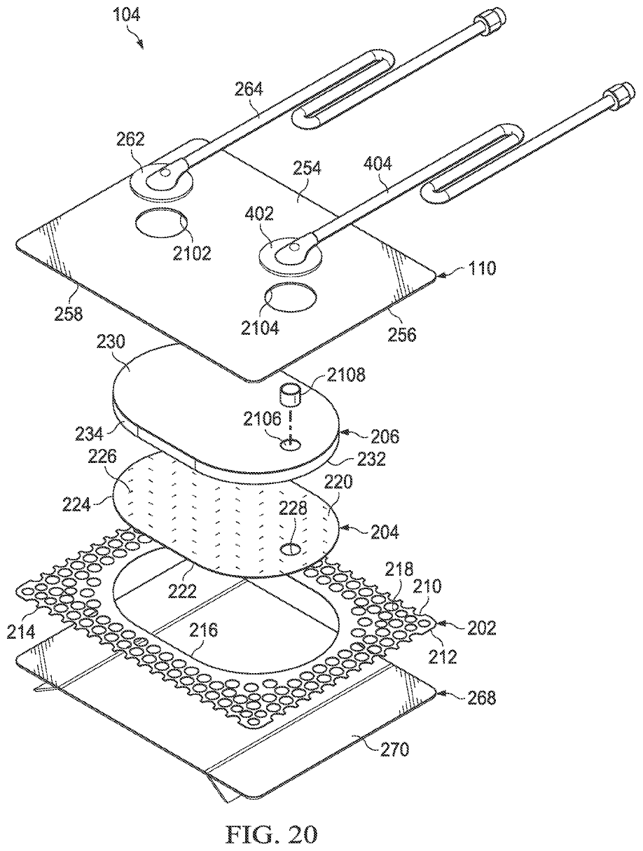

FIG. 20 is an exploded view of an example of the dressing 104 of FIG. 1, illustrating additional details that may be associated with some embodiments. In various implementations, the dressing 104 of FIG. 20 may be substantially similar to the dressing 104 of FIGS. 2-4 and 19, except that the first film layer 204 does not include the aperture 228, the manifold layer 206 is formed from an open-celled foam, the second film layer 208 is omitted, and the cover does not include the central aperture 260. In various implementations, the open-cell foam manifold layer 206 may have a height in a range of about 50 millimeters to about 250 millimeters, such as a height of about 50 millimeters, 100 millimeters, 150 millimeters, 200 millimeters, or 250 millimeters. In various implementations, the cover 110 may include a negative-pressure passage, such as an aperture 2102, and a fluid-ingress passage, such as aperture 2104. In various implementations, the adhesive layer 1903 may coat substantially all of the bottom surface 256 of the cover 110. When assembled, at least a portion of the bottom surface 256 of the cover 110 may be coupled to at least a portion of the top surface of the manifold layer 206 by the adhesive layer 1903, and at least another portion of the bottom surface 256 of the cover 110 may be coupled to a region of the sealing layer 202 between the periphery 214 and the treatment aperture 216. In various implementations, at least a portion of the top surface 220 of the first film layer 204 may be coupled to at least a portion of the bottom surface 232 of the manifold layer 206.

A fluid-ingress passage, such as aperture 2106, may be formed through and extend from the top surface 230 to the bottom surface 246 of the manifold layer 206. A fluid isolating member, such as fluid isolating member 2108, may be received within the aperture 2106 and/or the first film layer 204. In various implementations, the fluid isolating member 2108 may be a cylindrical body having a hollowed out interior portion, such as a tube. The fluid isolating member 2108 may have a height extending from the top surface 230 of the manifold layer 206 to the bottom surface 222 of the first film layer 204. In various implementations, an outer diameter of the fluid isolating member 2108 may be less than the diameter of the aperture 2106 and the diameter of the aperture 228, and an inner diameter of the fluid isolating member 2108 may be less than the outer diameter. In assembled form, the aperture 228, aperture 2106, fluid isolating member 2108, and aperture 2104 may be substantially coaxial.

FIG. 21 is an isometric view of an assembled example of the dressing 104 of FIG. 20 with dressing interfaces for negative-pressure wound therapy and instillation and/or oxygen therapy attached. As shown in FIG. 21, in some examples, the dressing interface 262 and the fluid conductor 264 may be attached to the top surface 254 and fluidly coupled to the interior of the dressing 104 through aperture 2102. Similarly, in some examples, the dressing interface 402 may be attached to the top surface 254 and fluidly coupled to the fluid-ingress passage through aperture 2104.

FIG. 22 is a cross-sectional view of the example dressing 104 of FIG. 21, taken at line 22-22, applied to the example tissue site 1902, and illustrating additional details associated with the therapy system 100 of FIG. 1. As illustrated in FIG. 22, in some examples, the bottom surface 256 of the cover 110 may be coated with an adhesive layer 1903, and at least a portion of the bottom surface 256 of the cover 110 may be coupled to at least a portion of the top surface 220 of the sealing layer 202 with the adhesive layer 1903, and at least a portion of the bottom surface 256 may be couped to at least a portion of the top surface 230 of the manifold layer 206. In various implementations, the aperture 228, aperture 2106, fluid isolating member 2108, and aperture 2104 may be coaxially aligned and define the fluid-ingress passage through the aperture 2104 and the interior or space inside of the inner diameter of the fluid isolating member 2108. In various implementations, the fluid-ingress passage is substantially isolated from the interior of the dressing 104, such as the spaces between the top surface 210 of the sealing layer 202 and the bottom surface 256 of the cover 110 occupied by the first film layer 204 and the manifold layer 206.

In operation, negative pressure may be provided to the wound 1904, and/or fluid may be removed from the wound 1904 by the negative-pressure source 102. For example, fluid may travel from the wound 1904 through at least one of the fluid passages 226 into the open cells of the foam manifold layer 206, and exit the dressing 104 to enter the dressing interface 262 through the aperture 2102. In operation, instillation solution and/or oxygen may enter the dressing 104 through the aperture 2104 from the dressing interface 402 and travel through the fluid-ingress passage defined by the aperture 2104, fluid isolating member 2108, and/or aperture 228 to enter the wound 1904 without entering the interior of the dressing 104. In various implementations, in order to ensure that the fluid isolating member 2108 effectively isolates the interior of the dressing 104 from the instillation solution and/or oxygen, the fluid isolating member 2108 may extend past the top surface 230 of the manifold layer 206 and touch the base of the dressing interface 402.

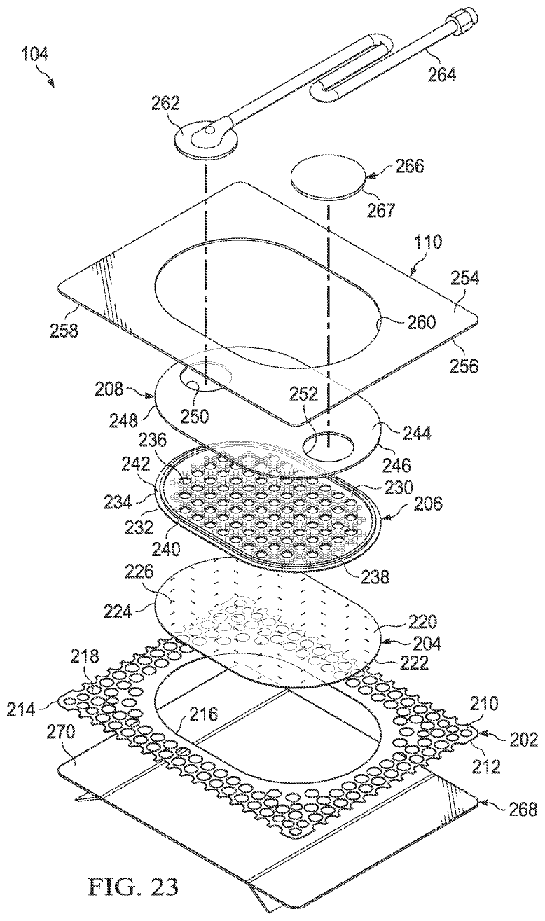

FIG. 23 is an exploded view of an example of the dressing 104 of FIG. 1, illustrating additional details that may be associated with some embodiments. In various implementations, the dressing 104 of FIG. 20 may be substantially similar to the dressing 104 of FIGS. 2-4 and 19, except that the first film layer 204 does not include the aperture 228 or the coupling region 241, the manifold layer 206 does not include the aperture 239 or the coupling region 243, and the second film layer 208 does not include the coupling region 253.

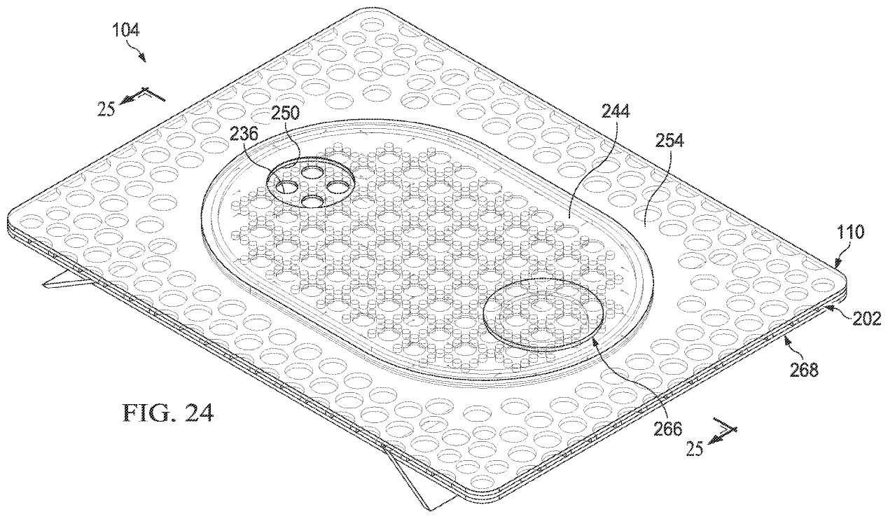

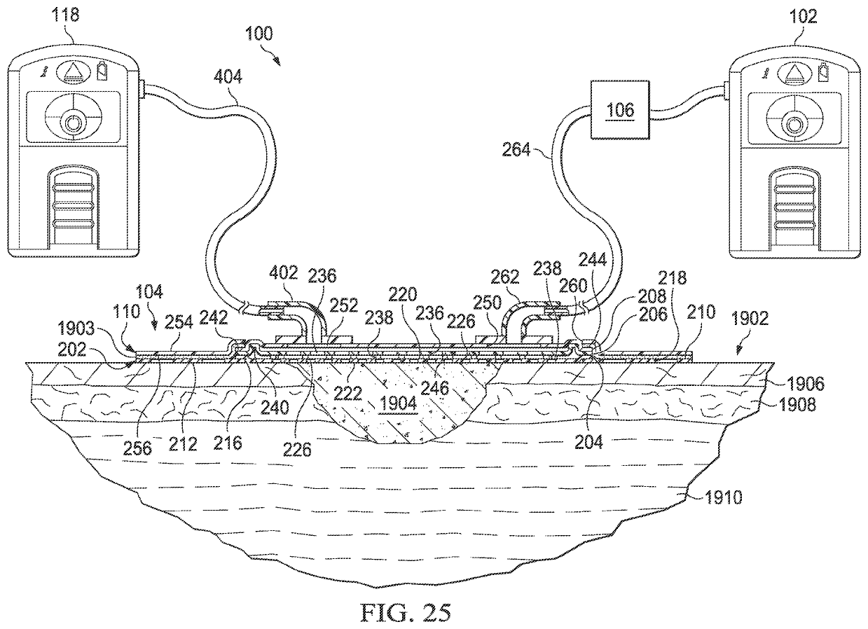

FIG. 24 is an isometric view of an assembled example of the dressing 104 of FIG. 23. FIG. 25 is a cross-sectional view of the example dressing of FIG. 24, taken at line 25-25, and applied to the example tissue site 1902. As shown in FIG. 25, in various implementations, the dressing interface 262 and the fluid conductor 264 may be attached to the top surface 254 and fluidly coupled to the interior of the dressing through aperture 250. In various implementations, the dressing interface 402 and the fluid conductor 404 may be attached to the top surface 254 and fluidly coupled to the interior of the dressing through aperture 252. In operation, instillation solution and/or oxygen may be provided to the wound 1904 and/or the periwound area via the dressing interface 402. In various implementations, instillation solution and/or oxygen may be provided by the dressing interface 402 to the aperture 252, and flow from the aperture 252 through the windows 236, from the windows 236 to the wound 1904 and/or the periwound area through the fluid passages 226.

The systems, apparatuses, and methods described herein may provide significant advantages. For example, conventional open-cell foam manifolds used in negative-pressure wound therapy systems may have free volumes in a range of about 50-90%. Instillation solution and/or oxygen may be typically provided to foam manifolds either after negative pressure has been released from the open-cell foam manifolds, or concurrently with the release of negative pressure (using the negative pressure at the manifold to draw instillation solution and/or oxygen into the manifold and tissue site until the pressure at the manifold reaches approximately ambient pressure). Thus, generally, the amount of instillation solution and/or oxygen required may be equal to the free volume of the open-cell foam manifold plus the volume required to be filled at the wound site. In conventional systems, a significant amount of instillation solution and/or oxygen may therefore be wasted in filling the free volume of the open-cell foam manifold, requiring larger negative-pressure wound therapy, instillation therapy, and/or oxygen therapy devices and systems. In addition, users may be required to manage greater volumes of instillation solution and/or oxygen. By providing fluid-ingress passages that bypass the interior of wound dressings and/or dressings having low interior volumes, the improved systems described herein may be able to significantly reduce the volume of instillation solution and/or oxygen that is required to treat a wound.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site with negative-pressure therapy, comprising:
a dressing configured to be placed adjacent to the tissue site, the dressing comprising:
a first film layer having a first side opposite a second side, a plurality of fenestrations disposed through the first side and the second side, the plurality of fenestrations configured to be exposed to the tissue site,
a manifold having a first side opposite a second side, the first side of the manifold disposed adjacent to the second side of the first film layer,
a second film layer having a first side opposite a second side, the first side of the second film layer disposed adjacent to the second side of the manifold,
a negative-pressure passage disposed through the second film layer in direct fluid communication between the second film layer and the manifold, and
a fluid-ingress passage disposed through the second film layer, the manifold, and the first film layer in direct fluid communication with the first side of the first film layer;
a negative-pressure source configured to be fluidly coupled to the negative-pressure passage; and
a fluid source configured to be fluidly coupled to the fluid-ingress passage.

2. The system of claim 1, wherein:
the first film layer further comprises a fluid-ingress aperture;
the manifold further comprises a fluid-ingress aperture;
the second film layer further comprises a fluid-ingress aperture; and
the fluid-ingress aperture of the first film layer, the fluid-ingress aperture of the manifold, and the fluid-ingress aperture of the second film layer are coaxially aligned to define the fluid-ingress passage through the fluid-ingress aperture of the first film layer, the fluid-ingress aperture of the manifold, and the fluid-ingress aperture of the second film layer.

3. The system of claim 2, wherein:
a portion of the second side of the first film layer around the fluid-ingress aperture of the first film layer is coupled to a portion of the first side of the manifold around the fluid-ingress aperture of the manifold; and
a portion of the first side of the second film layer around the fluid-ingress aperture of the second film layer is coupled to a portion of the second side of the manifold around the fluid-ingress aperture of the manifold.

4. The system of claim 3, wherein:
a periphery of the second side of the first film layer is coupled to a periphery of the first side of the manifold; and
a periphery of the first side of the second film layer is coupled to a periphery of the second side of the manifold.

5. The system of claim 4, wherein:
a first chamber is defined between the second side of the first film layer and the first side of the manifold, the portion of the second side of the first film layer coupled to the portion of the first side of the manifold, and the periphery of the second side of the first film layer coupled to the periphery of the first side of the manifold; and
a second chamber is defined between the first side of the second film layer and the second side of the manifold, the portion of the first side of the second film layer coupled to the portion of the second side of the manifold, and the periphery of the first side of the second film layer coupled to the periphery of the second side of the manifold.

6. The system of claim 5, wherein the first chamber and the second chamber are fluidly isolated from the fluid-ingress passage.

7. The system of claim 6, wherein the manifold further comprises a plurality of windows formed through the manifold.

8. The system of claim 7, wherein the first chamber is in fluid communication with the second chamber through the plurality of windows.

9. The system of claim 8, wherein the first side of the first film layer is configured to be in direct contact with the tissue site.

10. The system of claim 8, wherein the fluid-ingress passage is configured to be in direct fluid communication with the tissue site.

11. The system of claim 8, wherein:
the second film layer further comprises a negative-pressure aperture; and
the negative-pressure passage is defined by a fluid pathway from the negative-pressure aperture to the second chamber, the negative-pressure passage being configured to be positioned in fluid communication with the tissue site through the plurality of windows, the first chamber, and the plurality of fenestrations.

12. The system of claim 1, further comprising a sealing layer comprising a treatment aperture and a plurality of bonding apertures around the treatment aperture, wherein the first side of the first film layer and the plurality of fenestrations are configured to be exposed to the tissue site through the treatment aperture.

13. The system of claim 12, further comprising a cover layer having a first side opposite a second side, a pressure-sensitive adhesive disposed on the first side of the cover layer, the pressure-sensitive adhesive coupling the cover layer to the second film layer and the sealing layer, a portion of the pressure-sensitive adhesive disposed adjacent to the plurality of bonding apertures.

14. The system of claim 1, wherein the first film layer comprises thicker portions between each fenestration of the plurality of fenestrations and thinner portions proximate each fenestration of the plurality of fenestrations.

15. (canceled)

16. (canceled)

17. The system of claim 1, further comprising a plurality of standoffs formed on one or both of the first side and the second side of the manifold.

18. The system of claim 1, wherein the manifold comprises an open-cell foam.

19. (canceled)

20. The system of claim 1, wherein the fluid source is configured to provide at least one of an instillation solution or oxygen to the dressing.

21. (canceled)

22. The system of claim 1, wherein the manifold is fluidly isolated from the fluid-ingress passage.

23. A system for treating a tissue site with negative-pressure therapy, comprising:
a dressing configured to be positioned adjacent to the tissue site, comprising:
a first film layer having a plurality of fenestrations and a fluid-ingress aperture,
a manifold having a plurality of windows and a fluid-ingress aperture, and
a second film layer having a negative-pressure aperture and a fluid-ingress aperture;
wherein the first film layer, the manifold, and the second film layer are assembled in a stacked relationship to define a first chamber between the first film layer and the manifold and a second chamber between the second film layer and the manifold;
wherein the fluid-ingress aperture of the first film layer, the fluid-ingress aperture of the manifold, and the fluid-ingress aperture of the second film layer define a fluid-ingress passage through the dressing; and
wherein the first chamber and the second chamber are fluidly isolated from the fluid-ingress passage.

24. The system of claim 23, further comprising:
a fluid source configured to be coupled to the fluid-ingress passage through the fluid-ingress aperture of the second film layer;
wherein the fluid source is configured to be in direct fluid communication with the tissue site through the fluid-ingress passage.

25. The system of claim 23, further comprising:
a negative-pressure source configured to be coupled to the negative-pressure aperture of the second film layer;
wherein the negative-pressure source is configured to provide negative-pressure to the tissue site through the plurality fenestrations of the first film layer, the first chamber, the plurality of windows of the manifold, the second chamber, and the negative-pressure aperture of the second film layer.

26. (canceled)

27. (canceled)

28. (canceled)

29. (canceled)
